(19) United States
(12) Patent Application Publication
(10) Pub. No.: US 2008/0222743 A1
(43) Pub. Date: Sep. 11, 2008

(54) RNA interference and disease resistance in avians

(75) Inventors: Leandro Christmann, Watkinsville, GA (US); Jeffrey C. Rapp, Athens, GA (US)

Correspondence Address: AVIGENICS, INC., 111 RIVERBEND ROAD, ATHENS, GA, 30605, US

(73) Assignee: AviGenics, Inc.

(21) Appl. No.: 11/799,253

(22) Filed: May 1, 2007

Related U.S. Application Data
(63) Continuation-in-part of application No. 11/210,165, filed on Aug. 23, 2005, now abandoned.
Application No. 11/210,165 is a continuation-in-part of application No. 10/926,707, filed on Aug. 25, 2004, now abandoned.
(60) Provisional application No. 60/640,203, filed on Dec. 29, 2004.

(51) Int. Cl. C12N 15/87; A01K 67/027

(52) U.S. Cl. 800/21; 800/19

(57) ABSTRACT

The invention relates to transgenic avians whose genome contains nucleotide sequences which encode therapeutic polynucleotides that correspond to one or more certain sequences in the genome of an avian pathogen.

### RELATED APPLICATION INFORMATION

This application claims priority to U.S. patent application Ser. No. 11/210,165, filed Aug. 23, 2005, the disclosure of which is incorporated in its entirety herein by reference, which claims the benefit of U.S. provisional application No. 60/640,203, filed Dec. 29, 2004, the disclosure of which is incorporated herein in its entirety by reference and is a continuation-in-part of U.S. patent application Ser. No. 10/926,707, filed Aug. 25, 2004, the disclosure of which is incorporated herein in its entirety by reference.

### FIELD OF THE INVENTION

The present invention relates generally to the fields of biochemistry, molecular biology, genetics and avian medicine. More particularly, the invention relates to certain polynucleotides and their use to provide avians with protection against pathogen-induced diseases.

### BACKGROUND OF THE INVENTION

The present invention provides compositions and methods useful for protecting avians from certain pathogens. For example, the invention relates to RNA interference (RNAi) directed against such pathogens. RNAi is believed to be effected by double-stranded RNA which results in the degradation of specific RNA, for example, mRNA of certain avian pathogens such as influenza in avians and Marek's disease virus. Certain aspects of such gene silencing are disclosed in, for example, WO 99/32619; WO 01/75164; U.S. Pat. No. 6,506,559; Fire et al., Nature (1998) 391:806-811; Sharp, Genes Dev. (1999) 13:139-141; Elbashir et al., Nature (2001) 411:494-498; and Harborth et al., J. Cell Sci. (2001) 114:4557-4565. The disclosures of these two WO publications, this US patent and these four journal articles are incorporated in their entirety herein by reference.

Certain RNAi pathways have been characterized in Drosophila and Caenorhabditis elegans. In addition, “small interfering RNA” (siRNA) polynucleotides that interfere with expression of specific polypeptides in higher eukaryotes such as mammals (including humans) have also been examined. See, for example, Tuschl, (2001) Chembiochem. 2:239-245; Sharp, (2001) Genes Dev. 15:485-490; Bernstein et al., (2001) RNA 7:1509-1521; Zamore, (2002) Science 296:1265-1269; Plasterk, (2002) Science 296:1263-1265; Zamore (2001) Nat. Struct. Biol. 8:746-750; Matzke et al., (2001) Science 293:1080-1083; Scadden et al., (2001) EMBO Rep. 2:1107-1111, the disclosures of which are incorporated in their entirety herein by reference.

According to a current non-limiting model, the RNAi pathway is initiated by ATP-dependent, processive cleavage of long dsRNA into double-stranded fragments known as siRNAs which are typically about 18-27 nucleotide base pairs in length. In Drosophila, an enzyme known as “Dicer” is responsible for the cleavage of the double-stranded RNA. Dicer belongs to the RNase III family of dsRNA-specific endonucleases. See, for example, WO 01/68836; Bernstein et al., (2001) Nature 409:363-366, the disclosures of which are incorporated in their entirety herein by reference. According to this non-limiting model, the siRNA duplexes are incorporated into a protein complex followed by ATP-dependent unwinding of the siRNA generating an active RNA-induced silencing complex (RISC). See, for example, WO 01/68836, the disclosure of which is incorporated in its entirety herein by reference. The RISC complex recognizes and cleaves target RNA that is complementary to a strand of the siRNA contained in the RISC complex, thus interfering with expression of the specific protein encoded by the target RNA.

Many diseases caused by viral or bacterial pathogens afflict certain avians raised for commercial purposes, such as for food production. Various interventions have been employed to reduce or eliminate the prevalence of such livestock diseases. Among the most common are the prophylactic use of antibiotics and vaccinations. There are several disadvantages to these types of prophylactic measures. For example, each bird must be treated individually one or more times during its lifespan requiring considerable expenditures in both manpower and consumable goods. In addition, there is concern that widespread use of antibiotics induces selection of resistant strains of bacteria. Thus, over time commercially produced avians may become prone to diseases caused by resistant bacterial strains. Furthermore, avian bacterial pathogens may directly infect humans which may allow for antibiotic resistant avian pathogens to become resistant human pathogens causing a potential threat to the state of public health.

### SUMMARY OF THE INVENTION

There remains a need for improved methods of providing resistance to avian pathogens. In particular, there is a need for providing pathogen resistance which avoids the administration of antibiotics and immunogens used in vaccinations. In addition, there is a need for compositions and methods that confer disease resistance in avians which can be propagated from one generation to the next without further intervention. The present invention meets these and more. Provided for are nucleotide sequences, for example, isolated nucleotide sequences, which include a coding sequence for one or more therapeutic polynucleotides. Without wishing to limit the scope of the invention, the therapeutic polynucleotides may facilitate RNA interference in an avian cell inhibiting the propagation and/or replication of avian pathogens. The therapeutic polynucleotides may include a nucleotide sequence complementary, or substantially complementary, to a nucleotide sequence in the genetic material of an avian pathogen, for example, RNA of an avian pathogen such as avian influenza (e.g., mRNA). In one embodiment, the therapeutic polynucleotide includes a nucleotide sequence that is at least 80% complementary to a nucleotide sequence in the genetic material of an avian pathogen (i.e., target sequence). For example, the nucleotide sequence of the therapeutic polynucleotide may be at least about 85% complementary to the target sequence in the genetic material of an avian pathogen or at least about 90% complementary to the target sequence in the genetic material of an avian pathogen or at least about 95% complementary to the target sequence in the genetic material of an avian pathogen or at least about 99% complementary to the target sequence in the genetic material of an avian pathogen. Examples of the avian pathogen include influenza virus (avian influenza virus, i.e., AI), for example, avian influenza virus A, and Marek's disease virus. In one embodiment, the nucleotide sequence of the therapeutic polynucleotide is 100% complementary to the target sequence in the genetic material of an avian pathogen. In the case of a hairpin shaped therapeutic polynucleotide, the nucleotides in the loop sequence of the hairpin are typically not considered when determining percent identity of the therapeutic polynucleotide to the target sequence.

In one aspect, the invention is directed to a transgenic avian (e.g., a transgenic chicken, a transgenic turkey, a transgenic duck or a transgenic quail) containing in its genome a recombinant nucleotide sequence encoding a therapeutic polynucleotide which includes a nucleotide sequence that is complementary to a conserved nucleotide sequence in the genetic material of influenza virus A. Typically the polynucleotide is an RNA molecule which may be encoded by a transgene in the genome of the transgenic avian. In one embodiment, the therapeutic polynucleotide facilitates RNA interference in a cell of the transgenic avian. That is, the polynucleotide can provide for RNA interference against influenza A virus in cells of the transgenic avian. In one particularly useful embodiment, the recombinant nucleotide sequence is integrated in a chromosome of a cell of the avian. The invention also includes methods of producing such transgenic avians.

In one embodiment, the methods include providing a recombinant nucleotide sequence which encodes a therapeutic polynucleotide. The therapeutic polynucleotide typically contains a nucleotide sequence substantially complementary to a conserved nucleotide sequence in genetic material of an avian influenza virus wherein the therapeutic polynucleotide facilitates RNA interference in a cell of the transgenic avian. Transgenic avian cells can be produced by introducing the recombinant nucleotide sequence into avian cells capable of developing into a mature avian and thereafter obtaining a mature transgenic avian from the avian cell.

In one embodiment, the therapeutic polynucleotide encoded in the genome of the transgenic avian which is active against influenza virus A includes a first nucleotide sequence and a second nucleotide sequence wherein the second nucleotide sequence has substantially the same length as the first nucleotide sequence and is substantially complementary to the first nucleotide sequence and the first nucleotide sequence hybridizes at least in part to the second nucleotide sequence.

In one embodiment, the second nucleotide sequence is longer than the first nucleotide sequence. For example, the second nucleotide sequence may be longer than the first nucleotide sequence by one nucleotide, two nucleotides, three nucleotides or four nucleotides.

In one embodiment, the therapeutic polynucleotide encoded in the genome of the transgenic avian is between about 10 nucleotides and about 200 nucleotides in length, for example, between about 15 nucleotides and about 35 nucleotides in length.

In one embodiment, the conserved nucleotide sequence in the genetic material of influenza virus A (i.e., the target sequence) is about 10 to about 50 nucleotides in length, for example, about 15 to about 25 nucleotides in length.

In one embodiment, a nucleotide sequence of the therapeutic polynucleotide in the genome of the transgenic avian is at least about 90% complementary to the conserved nucleotide sequence in genetic material of influenza virus A. For example, the nucleotide sequence may be at least about 95% or may be may be at least about 96% or may be at least about 98% complementary to the conserved nucleotide sequence in genetic material of influenza virus A or may be identical to the conserved nucleotide sequence in genetic material of influenza virus A.

In one embodiment, the therapeutic polynucleotide is present in a cell of the avian and is included in a complex (e.g., a RISC complex) which facilitates cleavage of a nucleotide sequence in genetic material of an avian influenza virus.

In one embodiment, the therapeutic polynucleotide encoded in the genome of the transgenic avian inhibits replication of the avian influenza virus. In one embodiment, the transgenic avians of the invention are protected against infection by influenza relative to an otherwise similar avian that is not transgenic.

In one particularly useful embodiment, the transgenic avian produces either sperm or ova comprising the recombinant nucleotide sequence encoding a therapeutic polynucleotide which includes a nucleotide sequence that is complementary to a conserved nucleotide sequence in genetic material of influenza virus A.

In one embodiment, the recombinant nucleotide sequence includes a promoter and/or an enhancer in operable relationship to the therapeutic polynucleotide sequence. For example, the enhancer and promoter can be in operable relationship providing for regulation of expression (i.e., transcription) of the therapeutic nucleotide sequence. Any useful promoter or enhancer may be employed in the recombinant nucleotide sequence, for example, a polymerase III promoter or a functional portion thereof may be employed.

In one aspect the nucleotide sequence substantially identical to a conserved nucleotide sequence is present in a coding sequence of NP, PA, PB1, PB2, M and NS of influenza virus A. For example, the conserved nucleotide sequence can be one or more DNA sequences corresponding to (T in place of U) SEQ ID NO: 23, SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; of SEQ ID NO: 34; SEQ ID NO: 35; SEQ ID NO: 36; SEQ ID NO: 37; SEQ ID NO: 38; SEQ ID NO: 39; SEQ ID NO: 40; SEQ ID NO: 41; SEQ ID NO: 42 and functional portions thereof. Typically, the transgenic avians of the invention will also have the complementary nucleotide sequence to the conserved nucleotide sequence in their genome.

In one embodiment, the therapeutic polynucleotide which is active against influenza virus A includes a first nucleotide sequence attached to a second nucleotide sequence by a loop sequence wherein the second nucleotide sequence has substantially the same length as the first nucleotide sequence and is substantially complementary to the first nucleotide sequence and typically the first nucleotide sequence hybridizes to the second nucleotide sequence to form a hairpin. In one embodiment, the second nucleotide sequence is longer than the first nucleotide sequence by one of, for example, two nucleotides, three nucleotides or four nucleotides.

In one embodiment, the transgenic avian contains in its genome a nucleotide sequence encoding a therapeutic polynucleotide comprising a nucleotide sequence complementary to a nucleotide sequence in genetic material of an avian influenza virus. In one embodiment, the therapeutic polynucleotide comprises a first nucleotide sequence attached to second nucleotide sequence by a loop sequence. In one embodiment, the second nucleotide sequence has substantially the same length as the first nucleotide sequence and is substantially complementary to the first nucleotide sequence.

The invention contemplates the production of double stranded RNA fragments in transgenic avians of the invention corresponding to each of the nucleotide sequences disclosed herein, for example, SEQ ID NOS: 1 to 42 and their complement. In one embodiment, the double stranded RNA fragments are joined at one end by a loop structure (e.g., shRNAs). In another embodiment, the double stranded RNA fragments are not joined by a loop structure (e.g., siRNAs).

In the case where the double stranded RNA fragments are not joined by a loop structure, two separate nucleotide sequences can be produced in a transgenic avian which anneal to produce the double stranded RNA fragments. For example, transgenic avians can be produced using a vector having two promoters, each promoter operably linked to a coding sequence for one of the two RNA sequences. In another example, transgenic avians can be produced having two vectors in their genome each containing a promoter linked to a coding sequence for one of the RNA sequences. Such birds can be produced as is understood in the art by, for example, crossing two lines, one having a transgene encoding one of the RNA sequences and the other line having a transgene encoding the other RNA sequence.

The therapeutic polynucleotides may include a nucleotide sequence identical, or substantially identical, to a nucleotide sequence in the genetic material of an avian pathogen, for example, RNA of an avian pathogen (e.g., mRNA). In one embodiment, the therapeutic polynucleotide includes a nucleotide sequence that is at least 80% identical to a nucleotide sequence in the genetic material of an avian pathogen (i.e., target sequence). For example, the nucleotide sequence of the therapeutic polynucleotide may be at least about 85% identical to the target sequence in the genetic material of an avian pathogen or at least about 90% identical to the target sequence in the genetic material of an avian pathogen or at least about 95% identical to the target sequence in the genetic material of an avian pathogen or at least about 99% identical to the target sequence in the genetic material of an avian pathogen. In one embodiment, the nucleotide sequence of the therapeutic polynucleotide is 100% identical to the target sequence in the genetic material of an avian pathogen such as influenza virus (e.g., influenza virus A) or Marek's disease.

In addition, the therapeutic polynucleotides may include a nucleotide sequence identical, or substantially identical, to a nucleotide sequence in the genetic material of an avian pathogen, for example, RNA of an avian pathogen (e.g., mRNA) and a nucleotide sequence complementary, or substantially complementary, to a nucleotide sequence in the genetic material of an avian pathogen (e.g., AI), for example, RNA of an avian pathogen (e.g., mRNA). In one embodiment, the therapeutic polynucleotide includes a nucleotide sequence that is at least 80% identical to a nucleotide sequence in the genetic material of an avian pathogen (i.e., target sequence) and a nucleotide sequence that is at least 80% complementary to a nucleotide sequence in the genetic material of an avian pathogen (i.e., target sequence). For example, the nucleotide sequences of the therapeutic polynucleotide may be at least about 85% identical and at least about 85% complementary to the target sequence in the genetic material of an avian pathogen or at least about 90% identical and at least about 90% complementary to the target sequence in the genetic material of an avian pathogen or at least about 95% identical and at least about 95% complementary to the target sequence in the genetic material of an avian pathogen or at least about 99% identical and at least about 99% complementary to the target sequence in the genetic material of an avian pathogen. In one embodiment, the nucleotide sequences of the therapeutic polynucleotide are 100% identical and 100% complementary to the target sequence in the genetic material of an avian pathogen such as AI or Marek's disease.

In another embodiment, the nucleotide sequence in the genetic material of an avian pathogen is included, or substantially included, in one or more of SEQ ID NO: 17, SEQ ID NO: 18, SEQ ID NO: 19, SEQ ID NO: 20, SEQ ID NO: 21, and SEQ ID NO: 22 or a portion thereof, the complement of SEQ ID NO: 17, SEQ ID NO: 18, SEQ ID NO: 19, SEQ ID NO: 20, SEQ ID NO: 21, and SEQ ID NO: 22 or a functional portion thereof.

In one aspect of the invention, the therapeutic polynucleotide is RNA. In one embodiment, the therapeutic polynucleotide is in single stranded form. Therapeutic polynucleotides may be useful to treat (e.g., prevent) more than one disease in an avian. For example, a single therapeutic polynucleotide may be useful to treat one or two or three or four or five or more diseases. For example, in one embodiment, a single polynucleotide is useful to treat Marek's disease virus and herpes virus of turkey.

The invention includes cells of a transgenic avian that contain a therapeutic polynucleotide (e.g., RNA) encoded by a transgene present in the cells of the transgenic bird. Although the invention is not limited thereto, the transgene is typically present in the genome of cells of the transgenic bird.

Therapeutic polynucleotides may be included in a complex, for example, a RISC complex, which may include genetic material of an avian pathogen. Being included in the complex may facilitate cleavage of a target nucleotide sequence in the genetic material of an avian pathogen.

In one aspect of the invention, the therapeutic polynucleotide includes a first nucleotide sequence attached to second nucleotide sequence with an intervening loop sequence. The second nucleotide sequence may have substantially the same length as the first nucleotide sequence and is typically substantially complementary to the first nucleotide sequence. Without wishing to limit the invention to any theory, it is believed that the first nucleotide sequence will typically hybridize to the second nucleotide sequence to form a hairpin, for example, in a cell of a transgenic avian. In one useful embodiment, the second nucleotide sequence is longer than the first nucleotide sequence by one nucleotide or two nucleotides or three nucleotides or four nucleotides or five nucleotides or more.

Examples of therapeutic polynucleotides of the invention include those encoded by SEQ ID NO: 4, SEQ ID NO: 6, SEQ ID NO: 8, SEQ ID NO: 10, SEQ ID NO: 12 and SEQ ID NO: 14, a functional portion of SEQ ID NO: 4, SEQ ID NO: 6, SEQ ID NO: 8, SEQ ID NO: 10, SEQ ID NO: 12 and SEQ ID NO: 14 and those encoded by the complement of SEQ ID NO: 4, SEQ ID NO: 6, SEQ ID NO: 8, SEQ ID NO: 10, SEQ ID NO: 12 and SEQ ID NO: 14, a functional portion of SEQ ID NO: 4, SEQ ID NO: 6, SEQ ID NO: 8, SEQ ID NO: 10, SEQ ID NO: 12 and SEQ ID NO: 14.

Therapeutic polynucleotides of the invention may be of any useful length. That is, the therapeutic polynucleotide may include any useful number of nucleotides. In one embodiment, the therapeutic polynucleotide is between about 10 nucleotides and about 200 nucleotides in length, for example, between about 15 nucleotides and about 100 nucleotides in length or between about 15 nucleotides and about 70 nucleotides in length or between about 15 nucleotides and about 35 nucleotides in length. In certain useful embodiments, the therapeutic polynucleotide is 15 nucleotides, or 16 nucleotides, or 17 nucleotides, or 18 nucleotides, or 19 nucleotides, or 20 nucleotides, or 21 nucleotides, or 22 nucleotides, or 23 nucleotides, or 24 nucleotides, or 25 nucleotides, or 26 nucleotides, 27 nucleotides, 28 nucleotides, 29 nucleotides or 30 nucleotides in length.

In one embodiment, nucleotide sequences of the invention include a vector. In one embodiment, the vector includes the coding sequence of a therapeutic polynucleotide. The vector may be circular or linear and may include, for example, and without limitation, a promoter and/or an enhancer in operable relationship to the therapeutic polynucleotide coding sequence. A promoter in operable relationship to a therapeutic polynucleotide coding sequence may be effective to express, i.e., transcribe, the therapeutic polynucleotide in a cell of a transgenic avian. An enhancer in operable relationship to a therapeutic polynucleotide coding sequence may be effective to facilitate expression of the therapeutic polynucleotide in an avian cell. Examples of promoters useful in the present invention include, without limitation, Pol III promoters (including type 1, type 2 and type 3 Pol III promoters) such as H1 promoters, U6 promoters, tRNA promoters, RNase MPR promoters and functional portions of each of these promoters. Other promoters that may be useful in the present invention include, without limitation, Pol I promoters, Pol II promoters, cytomegalovirus (CMV) promoters, rous-sarcoma virus (RSV) promoters, murine leukemia virus (MLV) promoters, mouse mammary tumor virus (MMTV) promoters, SV40 promoters, ovalbumin promoters, lysozyme promoters, conalbumin promoters, ovomucoid promoters, ovomucin promoters, ovotransferrin promoters and functional portions of each of these promoters. Typically, functional terminator sequences are selected for use in the present invention in accordance with the promoter that is employed.

Therapeutic polynucleotide sequences such as those encoding siRNAs (e.g., shRNAs) operably linked to promoters can be cloned in tandem into a viral vector such as an avian retroviral vector. In one embodiment, spacer sequences are inserted between each shRNA expression cassette in order to minimize transcription interference between the two cassettes. In one embodiment, each shRNA coding sequence is under the control of an individual promoter. For example, vectors in which transcription of shRNA sequences from one or more of NP, PB1 and PA are each driven by a separate promoter are contemplated for use in producing disease resistant avians.

In one embodiment, the isolated nucleotide sequences of the present invention are contemplated as being introduced into and existing in a cell of a transgenic avian. In one embodiment, an isolated nucleotide sequence is integrated in a chromosome of an avian cell which may be present in a transgenic avian. In one useful embodiment, the cell is a germ-line cell. For example, the cell may be a germ-line cell present in a transgenic avian.

The invention also provides for methods of producing transgenic avians which include an isolated nucleotide sequence of the invention. Any useful method, such as those well known in the art, may be employed to produce the transgenic avians. In one embodiment, the transgenic avians are obtained from transgenic avian cells, produced as described herein, capable of developing into a mature avian. In one embodiment, the transgenic avian produces either sperm or ova which includes the coding sequence for a therapeutic polynucleotide. In one useful embodiment, the transgenic avian is protected against infection by an avian pathogen relative to a substantially similar avian, for example, an identical avian, that does not comprise an isolated nucleotide sequence of the invention.

Avians as disclosed herein include, without limitation, chicken, quail, turkey, duck, goose, pheasant, parrot, finch, hawk, crow, ratite including ostrich, emu and cassowary. In one useful embodiment, the avian is a chicken, turkey or duck.

Any combination of features described herein is included within the scope of the present invention provided that the features included in any such combination are not mutually inconsistent. Such combinations will be apparent to one of ordinary skill in the art.

### BRIEF DESCRIPTION OF THE DRAWINGS

FIG. 1 (A, B, C and D) shows the activity of an interfering LTR promoter before and after inactivation.

FIG. 2 (A, B and C) show the construction of a shRNA retroviral vector. FIG. 2 A shows the structure of the double-stranded oligonucleotide used to construct shRNA based upon RNA sequence of SEQ ID NO: 35. FIG. 2 B shows the structure of the shRNA after transcription from an integrated retroviral vector. FIG. 2 C shows the structure of the shRNA retroviral vector. “LTR” stands for long terminal repeat and “neo” stands for neomycin resistance gene.

### DEFINITIONS

Certain terms employed in the present patent application are defined below.

The term “avian” as used herein refers to any genus, species, subspecies or strain of organism of the taxonomic class ava, such as, but not limited to chicken, turkey, duck, goose, quail, pheasant, parrot, finch, hawk, crow, ratite including ostrich, emu and cassowary. The term includes the various known strains of Gallus gallus, or chickens, (for example, White Leghorn, Brown Leghorn, Barred-Rock, Sussex, New Hampshire, Rhode Island, Australorp, Minorca, Amrox, California Gray), as well as strains of turkey, pheasant, quail, duck, ostrich and other poultry commonly bred in commercial quantities. It also includes an avian organism in each stage of development, including embryonic and fetal stages. The term “avian” also may denote “pertaining to an avian”, such as “an avian cell.”

As used herein the term “avian pathogen” and similar terms and phrases relate to any viral or bacterial pathogen that may infect an avian. A viral pathogen may have a DNA genome or it may have an RNA genome.

As used herein, the terms “complement”, “complementary” and “complementarity” and similar terms and phrases relate to a nucleotide sequence or nucleotide sequences whose bases form intermolecular and/or intramolecular base pairs as conventionally understood by workers of skill in fields such as molecular biology and genomics. The degree of complementarity of two sequences can be quantified and the sequences can be, for example, at least about 75% complementary to each other, or at least about 80% complementary to each other, or at least about 85% complementary to each other, or at least about 90% complementary to each other, or at least about 95% complementary to each other, or at least about 96% complementary to each other, or at least about 98% complementary to each other, or at least about 99% complementary to each other, or 100% complementary to each other.

The term “functional portion” or “functional fragment” as used herein refers to a portion of a specified molecule or complex which is capable of substantially or detectably performing the function of the specified molecule or complex. For example, a functional fragment of a specified nucleotide sequence is capable of substantially or detectably performing the function of the specified nucleotide sequence, such as the function of performing RNA interference.

“Genetic material” refers to nucleic acid included in or encoded by an organism capable of reproduction such as an animal, a cell or a virus. For example, genetic material includes all DNA or RNA in the genome of the organism, all DNA or RNA that may be produced based on the sequence of DNA or RNA in the genome of the organism.

The term “heterologous” as it relates to nucleic acid sequences includes nucleotide sequences that are not normally associated with a particular chromosomal locus and/or are not normally associated with a particular cell type or organism type. For example, a host cell transformed with a vector which is not normally present in the host cell would be considered to be heterologous for purposes of this invention.

A “nucleoside” is conventionally understood by workers of skill in fields related to the present invention as comprising a monosaccharide linked in glycosidic linkage to a purine or pyrimidine base. A “nucleotide” comprises a nucleoside with at least one phosphate group appended, typically at a 3′ or a 5′ position (for pentoses) of the saccharide, but may be at other positions of the saccharide. A nucleotide may be abbreviated herein as “nt.” Nucleotide residues occupy sequential positions in an oligonucleotide or a polynucleotide. Accordingly a modification or derivative of a nucleotide may occur at any sequential position in an oligonucleotide or a polynucleotide. All modified or derivatized oligonucleotides and polynucleotides are encompassed within the invention and fall within the scope of the claims. Modifications or derivatives can occur in the phosphate group, the monosaccharide or the base.

By way of nonlimiting examples, the following descriptions provide certain modified or derivatized nucleotides. The phosphate group may be modified to a thiophosphate or a phosphonate. The phosphate may also be derivatized to include an additional esterified group to form a triester. The monosaccharide may be modified by being, for example, a pentose or a hexose other than a ribose or a deoxyribose. The monosaccharide may also be modified by substituting hydroxyl groups with hydro or amino groups, by esterifying additional hydroxyl groups. The base may be modified as well. Several modified bases occur naturally in various nucleic acids and other modifications may mimic or resemble such naturally occurring modified bases. Nonlimiting examples of modified or derivatized bases include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Nucleotides may also be modified to harbor a label. Nucleotides having a fluorescent label or a biotin label, for example, are available from Sigma (St. Louis, Mo.).

As used herein the terms “% identical”, “percent identical”, “percent identity”, and similar terms and phrases relate to a position-by-position comparison between one or more molecular sequences, for example, comparison between a first sequence or subsequence and a second sequence or subsequence. The comparison determines the percent of the positions in the two sequences which are identical to each other.

Nucleotide sequences that are 100% or less identical to each other may be similar or homologous sequences. The “degree of homology” and the “percent similarity” are synonymous to the terms “percent of identity” and “percent identity” and can refer to two sequences displaying at least about 75% identity, or at least about 80% identity, or at least about 85% identity, or at least about 90% identity, or at least about 95% identity, or at least about 96% identity, or at least about 98% identity, or at least about 99% identity to each other.

In one embodiment, the percent identity may be readily determined by comparing sequences of therapeutic polynucleotides or therapeutic polynucleotide coding sequences to the corresponding portion of a target sequence using any useful method, for example, using computer algorithms well known to those having ordinary skill in the art, such as Align or the BLAST algorithm (Altschul, J. Mol. Biol. 219:555-565, 1991; Henikoff and Henikoff, Proc. Natl. Acad. Sci. USA 89:10915-10919, 1992).

As used herein, the terms “operable relationship”, “operably linked”, and similar terms and phrases relate to the mutual juxtaposition of a transcription regulatory element, such as a promoter or enhancer, and a transcribable nucleotide sequence. Transcription regulatory elements are operably linked to a transcribable sequence when the transcribable sequence is positioned relative to, for example, linked to, the regulatory element in a manner that allows for or facilitates transcription of the transcribable sequence, for example in a host cell. The term “regulatory element” is intended to include promoters, enhancers and other transcription controlling elements such as polyadenylation signals. Such regulatory sequences are described, for example, in Goeddel (1990) Gene Expression Technology: Methods in Enzymology, 185, Academic Press, San Diego, Calif., the disclosure of which is incorporated in its entirety herein by reference. Regulatory sequences include those that direct constitutive or non-constitutive transcription of a nucleotide sequence in any useful cell and those that direct transcription of a nucleotide sequence in certain host cells, such as tissue-specific regulatory sequences.

The terms “influenza virus” or “avian influenza virus” and AI are used interchangeably herein and refer to RNA viruses of the family Orthomyxoviridae which are carried by birds and in some instances can be transmitted to humans.

The term “polynucleotide” and similar terms and phrases such as “polynucleotide sequence” or “nucleotide sequence” are used as conventionally understood by workers of skill in fields such as biochemistry, molecular biology, genomics, and similar such fields. For example, the meaning of polynucleotide is understood to include nucleotide polymers. A polynucleotide employed in the present invention may be single stranded or it may be a base paired double stranded structure or a triple stranded base paired structure. A polynucleotide may be a DNA, an RNA or any useful mixture or combination of a DNA strand and an RNA strand, such as, by way of nonlimiting example, a DNA-RNA hybrid duplex structure. In addition, a polynucleotide can include one or more strands which include a mixture of nucleotides such as ribonucleotides and deoxyribonucleotides. A polynucleotide is typically, though not exclusively, about 10 nucleotides or base pairs in length or longer. In view of the size of many polynucleotides envisioned in the present invention; the polynucleotides may be termed “oligonucleotides” by workers of skill in fields related to the present invention. Nucleotide sequences disclosed herein, whether RNA sequences or DNA sequences, are disclosed using the letters G, A, T or C. Therefore, T is typically used in an RNA sequence to indicate uracil and in a DNA sequence to indicate thymadine.

As used herein a “promoter” and similar terms and phrases relate to a site on a DNA at which transcription of a nucleotide coding sequence is initiated. The promoter may be modified by the addition, deletion or substitution of nucleotide sequences while maintaining a functional promoter. Many eukaryotic promoters include two types of recognition sequences: the TATA box and the upstream promoter elements. The TATA box, located upstream of the transcription initiation site, is involved in directing RNA polymerase to initiate transcription at the correct site, while the upstream promoter elements may determine the rate of transcription and may be upstream of the TATA box. As used herein, “enhancer” elements can stimulate transcription from linked promoters.

An avian containing a transgene that is passed on to progeny avians is a “propagatable” transgenic avian.

“Substantially” as used herein, typically, means at least about 80%. For example, a nucleotide sequence that is substantially identical to another nucleotide sequence is at least about 80% identical to the other nucleotide sequence.

A “conserved nucleotide sequence” refers to a nucleotide sequence that has been determined to be greater that 90% conserved among known variants of the gene obtained from strains of a particular organism such as strains of influenza type A virus.

As used herein the term “target sequence” and similar terms and phrases relate to a nucleotide sequence that occurs in the genetic material, for example, RNA, of an avian pathogen against which a polynucleotide, for example, a therapeutic polynucleotide, of the invention is directed. A “targeting sequence” of a therapeutic polynucleotide is a nucleotide sequence directed against a sequence contained within the genetic material of an avian pathogen, i.e., target sequence. A targeting sequence typically includes a sequence that is substantially identical to the target sequence and/or a sequence whose complement is substantially identical to the target sequence. By targeting a pathogen sequence, polynucleotides of the invention, for example, therapeutic polynucleotides, can have the ability to initiate RNA interference.

The term “therapeutic polynucleotide” refers to a polynucleotide of the present invention useful to prevent or treat diseases, such as avian diseases. A therapeutic polynucleotide includes a nucleotide sequence which is at least partially complementary to a target sequence of a pathogen. Certain therapeutic polynucleotides may be about 65%, about 70%, about 75%, about 80%, about 85%, about 90%, about 95%, about 99% or about 100% complementary to the target sequence. In one embodiment, a therapeutic polynucleotide is referred to as a targeting sequence.

As used herein, the term “transformation” refers to the introduction of foreign nucleic acid, for example, DNA, into a host cell. Transformation can be accomplished by any useful method. Techniques for transformation may include, without limitation, calcium phosphate or calcium chloride co-precipitation, DEAE-dextran-mediated transfection, lipofection, infection by recombinant viral vectors, ballistic particle projection, microinjection, electroporation or combinations thereof. Some suitable methods for transforming cells can be found in Sambrook, et al. (2001), Ausubel et al. (2002), and other laboratory manuals.

As used herein the term “transgene” and similar terms relate to a nucleotide sequence that has been incorporated into a cell, for example, a cell of an avian, through human manipulation. As used herein the term “transgenic” and similar terms when used to describe an avian relate to an avian at least some of whose cells include a transgene. Often, though not always, a transgene includes a heterologous nucleotide sequence. A transgene may be introduced into a cell using any useful method of cellular transformation. The transgene is stably incorporated in the genome of a cell if the transgene is passed from the host cell to progeny cells during mitotic or meiotic cell division.

Transgenes contemplated in the present invention include nucleotide sequences which are complementary to a target sequence of an avian pathogen or are identical or homologous to a target sequence of an avian pathogen. In one embodiment, the transgene which includes a nucleotide sequence encoding a therapeutic polynucleotide, an operably linked promoter and/or enhancer is incorporated into the genomic DNA of an avian cell. In one embodiment, a genomically incorporated transgene is stably incorporated and is passed on to progeny cells by mitotic or meiotic cell division. In particular, as a result of stable incorporation into germ line cells, meiotic cell division results in the transgene being passed on to progeny avians.

As used herein, a “transgenic avian” is an avian in which one or more of the cells of the avian includes a transgene. A transgene is typically heterologous DNA that may be integrated into the genome of a cell from which a transgenic avian has developed and that remains in the genome of the mature avian where it directs the transcription of a transgene coding sequence, for example, a therapeutic polynucleotide coding sequence, in one or more cell types or tissues of the transgenic avian.

A “vector”, as used herein, generally refers to a nucleic acid molecule capable of transporting into a cell a transgene which includes a nucleotide sequence comprising a therapeutic polynucleotide coding sequence. One type of vector is a “plasmid” which refers to a circular double stranded DNA molecule into which nucleotide sequences of interest can be inserted. Another type of vector is a viral vector, wherein DNA segments can be inserted into the viral genome. Some vectors are capable of autonomous replication in a host cell into which they are introduced, such as, bacterial vectors and episomal mammalian vectors. Other vectors are designed to integrate into the genome of a host cell and are thereafter replicated with replication of the host genome.

Vectors may include, without limitation, any of the following elements: an origin of replication, a promoter, an enhancer, a cassette or insert, coding sequences for a 5′ mRNA leader sequence, a ribosomal binding site, a transcription termination site, a polyadenylation coding site and selectable marker sequences. Typically, in a vector, the cassette contains the nucleic acid sequence to be expressed. Vectors typically facilitate the manipulation or transfer of genetic material, for example, from one organism to another. A vector or plasmid may be single stranded, double stranded, linear, open circular, or supercoiled DNA or RNA.

A vector may be constructed so that a particular nucleotide sequence, such as a therapeutic polypeptide of the invention, is located in the vector and positioned relative to certain regulatory sequences included in the vector, such as a promoter, so that the coding sequence is transcribed under the control of the regulatory sequences, i.e., operably linked to the regulatory sequences.

### DETAILED DESCRIPTION OF THE INVENTION

The present invention provides compositions and methods useful for protecting avians from certain pathogens. For example, the invention relates to RNA interference (RNAi) directed against such pathogens.

In one embodiment, the present invention provides compositions and methods which relate to protecting an avian from infection by avian pathogens by stably incorporating nucleotide sequences into the genome of avian cells, for example, avian germ-line cells. The nucleotide sequences are specifically directed against one or more pathogen target sequences and are typically unrelated to nucleotide sequences present in the native or unmodified genome of the avian.

The nucleotide sequences of the invention are capable of inhibiting the growth or replication of an avian pathogen within an avian cell. In one useful embodiment, the pathogen is a virus, for example, influenza virus such as influenza types A (e.g., H5N1 and related AI strains), B and C. In one embodiment, useful nucleotide sequences for production of siRNAs and shRNA in transgenic avians such as chickens as disclosed herein are disclosed in US patent publication No. 2005/0008617, published Jan. 13, 2005, the disclosure of which is incorporated in its entirety herein by reference. For example, each of the siRNAs and shRNAs sequences, and their complement, and each of the siRNAs and shRNAs coding sequences, and their complement, disclosed in US patent publication No. 2005/0008617, such as SEQ ID NOS: 1 to 32 of US patent publication No. 2005/0008617, are contemplated for use in accordance with the present invention.

In another embodiment, the avian pathogen is Marek's disease virus (MDV) or a related virus such as herpes virus of turkey (HVT). Marek's disease virus may be, for example, Marek's disease virus Types 1, 2, or 3 or other certain variants or strains of Marek's disease virus, for example, MDV strain Md5, which has been sequenced by Tulman, et al., (2000) J. Virol. 74:7980-7988. In one embodiment, the target sequence is encoded by the transcript of the Marek's disease virus homologue of ICP4. The sequence of the Marek's disease virus homologue of the ICP4 gene is disclosed in Anderson et al (1992) Virology 189:657-667. The disclosures of Tulman et al and Anderson et al are incorporated in their entirety herein by reference.

Also contemplated for use as disclosed herein are nucleotide sequences that hybridize to nucleotide sequences disclosed herein. Examples of sequences disclosed herein include the nucleotide sequences disclosed in SEQ ID NO: 1 to SEQ ID NO: 42. In one embodiment, nucleotide sequences disclosed herein and nucleotide sequences that can hybridize to nucleotide sequences disclosed herein are used as hybridization probes.

In one embodiment, hybridizations are under stringent conditions, for example, medium stringency conditions or high stringency conditions. High stringency conditions, when used in reference to nucleic acid hybridization, may comprise conditions equivalent to binding or hybridization at 65° C. in a solution consisting of 6×SSPE, 1% SDS, 5×Denhardt's reagent and 100 μg/ml denatured salmon sperm DNA followed by washing in a solution comprising 0.1×SSPE, and 0.1% SDS at 65° C. for about 15 to about 20 minutes. In certain embodiments, the wash conditions may include 50% formamide at 42° C. instead of 65° C. High stringency washes may include 0.1×SSC to 0.2×SSC and 1% SDS at 65° C. for about 15 to about 20 min. (see, Sambrook et al., Molecular Cloning—A Laboratory Manual (2nd ed.) Vol. 1-3, Cold Spring Harbor Laboratory, Cold Spring Harbor Press, N.Y., 1989, the disclosure of which is incorporated herein in its entirety by reference). Exemplary medium stringency conditions are as described above for high stringency except that the washes are carried out at 55° C. or at 37° C. when in the presence of 50% formamide.

Other important viral pathogens from which avians may be protected in accordance with compositions and methods disclosed herein include, without limitation, all groups, variants and serotypes of avian adenovirus, including avian adenovirus Group I (CELO), avian adenovirus Group II (HEV) and avian adenovirus Group III (EDS-76); avian encephalomyelitis; avian nephritis; avian reovirus; avian rhinotracheitis including avian rhinotracheitis UK and HG; chicken anemia virus; fowl pox virus; lymphoid leucosis virus, including lymphoid leucosis virus Groups A, B, C, D; Newcastle disease virus; paramyxovirus, including paramyxovirus Type 2; reticuloendotheliosis virus and the causative agents for infectious bronchitis, including infectious bronchitis; infectious bursal disease and infectious laryngotracheitis.

Prevention and treatment of infections by avian bacterial pathogens are also contemplated in the present invention. Such bacterial pathogens include, without limitation, Mycobacterium avium, Haemophilus paragallinarum, Mycoplasma gallisepticum, Mycoplasma synoviae, Salmonella gallinarum/pullorum and other Salmonella species.

The genomes of all variants, mutants, strains, serotypes and evolved or selected subspecies of viral and bacterial avian pathogens disclosed herein are contemplated as including nucleotide sequences encoding target sequences for the purposes of the present invention. In particular, it is understood among workers of ordinary skill in the fields of virology, bacteriology, microbiology, avian veterinary medicine, and similar fields related to the present invention that a viral or bacterial pathogen evolves in response to host changes and to therapeutic interventions administered to avians, as well as in more general ways, during the lifetime of all patents that issue of this invention. Therefore, all variants, mutants, strains, serotypes and subspecies of viral and bacterial avian pathogens that may arise during the lifetime of such patents comprise equivalent target sequences for the purposes of the present invention. Such equivalent target sequences are included within the scope of the claims.

In one aspect of the invention, nucleotide sequences encode therapeutic polynucleotides involved in targeting genetic material of avian pathogens. Without wishing to limit the invention to any particular theory or mechanism of operation, it is believed that therapeutic polynucleotides assemble with cellular proteins into an endonuclease complex referred to as an RNA-induced silencing complex (RISC). The RISC exhibits sequence specific endoribonuclease activity directed against a target RNA sequence of the pathogen. In one embodiment, a therapeutic polynucleotide acts as a guide restricting the RISC to cleave only RNAs substantially complementary to a portion of the therapeutic polynucleotide and/or substantially identical to a portion of the therapeutic polynucleotide.

The present invention contemplates the targeting of any RNA involved in avian pathogen propagation, for example, RNA involved in the propagation of Marek's disease virus or related viruses. Examples of targeted transcripts include, without limitation, pathogen gene transcripts encoding water soluble and non-water soluble proteins including transcripts encoding structural proteins and non-structural proteins.

Target transcripts may include RNA involved in the regulation of gene expression. Non-limiting examples of regulators of pathogen gene expression include, without limitation, positive or negative regulating factors, for example, transcription factors, kinases or phosphatases, cis or trans activating factors and polypeptides involved in pathogen biosynthetic or regulatory pathways.

Any segment of an RNA, for example, mRNA, employed in the propagation or survival of avian pathogens may be targeted in accordance with the present invention including, but not limited to, the 5′ untranslated (UT) region, the ORF and/or the 3′ UT region of the transcript.

In one embodiment, two or more independent therapeutic polynucleotides are used to reduce or eliminate the effects of a single pathogen. For example, two or more independent therapeutic polynucleotides can be employed to target the same gene transcript of a pathogen or two or more independent therapeutic polynucleotides can be employed to target different transcripts of the same pathogen.

One useful approach to produce an anti-pathogenic effect in accordance with the present invention is by the use of therapeutic polynucleotides comprising short interfering RNAs (siRNAs) or micro RNAs. At least a portion of each therapeutic polynucleotide siRNA is substantially complementary to at least a portion of the pathogen gene transcript target sequence or is substantially identical to at least a portion of the pathogen gene transcript target sequence. In addition, the siRNAs may include a nucleotide sequence substantially complementary to at least a portion of the pathogen gene transcript target sequence and a nucleotide sequence substantially identical to at least a portion of the pathogen gene transcript target sequence. See, for example, WO99/32619, WO01/75164, WO01/92513, WO 01/29058, WO01/89304, WO02/16620, and WO02/29858. The disclosures of each of these WO patent applications are incorporated by reference herein in their entirety.

To predict with certainty which siRNA sequences will in fact exhibit a desired effect, individual specific candidate siRNA polynucleotide or oligonucleotide sequences can be generated and tested to determine whether interference with expression of a desired polypeptide target is effected.

In accordance with the present invention, therapeutic polynucleotides such as siRNAs are designed based on the known nucleotide sequence of a portion of a pathogen genome. Design parameters for therapeutic polynucleotides are well known in the art and include, for example, those disclosed in Elbashir et al. (2001) Nature 411:494-498; and Elbashir et al. (2001) EMBO J. 20:6877-6888. The disclosures of each of these two references are incorporated herein in their entirety by reference. In one embodiment, therapeutic polynucleotides such as siRNA useful for RNA interference may be designed based on the following:
1. Select a region from a given cDNA sequence beginning 50-100 nt downstream of start codon;
2. Search for 15 to 40 nt sequence motif with of AA(Nₓ), for example a 21 nt sequence motif of AA(N₁₉);
3. Or search for 15 to 40 nt sequence motif of NA(Nₓ), for example, a 23-nt sequence motif NA(N₂₁) and convert the 3′ end of the sense siRNA to TT;
4. Or search for a 15 to 40 nt sequence motif of NAR(Nₓ)YNN, for example, NAR(N₁₇)YNN; and
5. The target sequence may optimally have a GC content of approximately 50%.
   A=Adenine; T=Thymine; R=Adenine or Guanine (Purines); Y=Thymine or Cytosine (Pyrimidines); N=any nucleotide.

TABLE 1

| | | Score | |
|---|---|---|---|
| Criteria | Description | Yes | No |
| 1 | Moderate to low (30%-52%) GC Content | 1 point | |
| 2 | At least 3 A/Us at positions 15-19 (sense) | 1 point/ | |
| | | per A or U | |
| 3 | Lack of internal repeats (Tm < 20° C.) | 1 point | |
| 4 | A at position 19 (sense) | 1 point | |
| 5 | A at position 3 (sense) | 1 point | |
| 6 | U at position 10 (sense) | 1 point | |
| 7 | No G/C at position 19 (sense) | | −1 point |
| 8 | No G at position 13 (sense) | | −1 point |

Table 1 lists 8 criteria and the methods of score assignment for certain therapeutic polynucleotide design. A sum score of 6 defines a potential cutoff for selecting siRNAs according to this particular method. That is, all siRNAs scoring higher than 6 may be useful therapeutic polynucleotides in this particular embodiment.

Computer programs are available which are useful to determine functional target nucleotide sequences. For example, publicly available programs such as the program available at http://.bioinfo2.clontech.com/maidesigner/ based on the method of Elbashir et al (2001) Genes and Development 15:188-200, the disclosure of which is incorporated in its entirety herein by reference, may be employed for such determinations. Other publicly available programs include http://design.rnai.jp/sidirect/index.php.

Standard experimental methodologies well known in the art may be used to confirm the efficacy of therapeutic polynucleotides identified using the above criteria.

The therapeutic polynucleotide or targeting sequence may be about 10 nucleotides (nt) in length to about 200 nt in length. In one embodiment, the length is about 15 to about 70 nt in length. For example, the therapeutic polynucleotide may be 16 nt, or 17 nt, or 18 nt, or 19 nt, or 20 nt, or 21 nt, or 22 nt, or 23 nt, or 24 nt, or 25 nt, or 26 nt, or 27 nt, or 28 nt, or 29 nt, or 30 nt in length.

The therapeutic polynucleotide or a portion thereof is typically at least about 80% complementary or at least about 80% identical to the pathogen sequence that it is targeting (i.e., target sequence). For example, in target sequences that are between about 16 nt and about 25 nt in length, typically there are no more than 3 or 4 or 5 nucleotides mismatched between the aligned portions of the therapeutic polynucleotide and the target sequence. The therapeutic polynucleotide or a portion thereof may be at least about 85% complementary or at least about 90% complementary or at least about 95% complementary or at least about 97% complementary or at least about 99% complementary or 100% complementary to the pathogen target sequence. In addition, the therapeutic polynucleotide or a portion thereof may be at least about 85% identical or at least about 90% identical or at least about 95% identical or at least about 97% identical or at least about 99% identical or 100% identical to the pathogen target sequence.

Certain useful therapeutic polynucleotides are sufficiently complementary to their target sequence such that they will hybridize with the target sequence or its complement under conditions within an avian cell.

In one embodiment, a complex is formed with the therapeutic polynucleotide that induces RNA interference promoting cleavage of the pathogen RNA. Any nucleotide sequence promoting such cleavage of an avian pathogen RNA falls within the scope of the present invention.

The present invention contemplates therapeutic polynucleotides which include a first nucleotide sequence complementary to a target sequence of an avian pathogen such as influenza virus (e.g., avian influenza virus type A), Marek's disease virus and related viruses such as turkey herpes virus, and a second nucleotide sequence complementary or substantially complementary to the first nucleotide sequence. See, for example, U.S. Pat. No. 6,506,559 and U.S. Pat. No. 6,531,647, the disclosures of which are incorporated in their entirety herein by reference. In one useful embodiment, the therapeutic polynucleotides are shRNAs. In one embodiment, the shRNA includes a first nucleotide sequence, an intervening loop-forming nucleotide sequence, and a second nucleotide sequence complementary or substantially complementary to the first nucleotide sequence. Without wishing to be bound by theory, it is believed that such a polynucleotide sequence including a first nucleotide sequence, a loop, and a second nucleotide sequence complementary or substantially complementary to the first nucleotide sequence, forms an intramolecular double stranded “hairpin” structure capable of producing an anti-pathogenic or therapeutic effect in an avian. The loop portion of the shRNA may be of any useful sequence. For example, any sequence may be employed which is not substantially self-complementary.

Avian influenza virus A RNAs encoded by genes NP, PA, PB1, PB2, M and NS are contemplated for targeting by RNAi as disclosed herein. Such RNAi targets have been disclosed for use against human influenza virus in Ge et al, PNAS (2003) 100: 2718-2723, the disclosure of which is incorporated in its entirety by reference herein.

Examples of siRNA sequences contemplated for production in transgenic avians which are designed to provide resistance to avian influenza virus A by RNA interference are SEQ ID NOS: 23 to 42, shown below. The complementary nucleotide sequence and target nucleotide sequences for each of these sequences can readily be deduced by a practitioner of skill in the art. The target sequences for each of these exemplary siRNA sequences are highly conserved among influenza A viral strains. In fact most of these sequences are reported as being 100% conserved among various influenza A viruses (see Gee et al (2003) PNAS vol 100, p 2718-2723). None of these exemplary sequences have more than one non-conserved nucleotide identified in their sequence.

The designation for each of the sequences indicates in what region of the genome of the influenza A virus the conserved sequence was identified. For example, sequences having a PA, PB1 and PB2 designation indicate sequence components of RNA transcriptase; sequences having a NP designation indicate sequence components of the nucleocapsid; sequences having an M designation indicate sequence components of structural proteins or proteins involved in the viral life cycle and sequences having an NS can indicate sequence components for certain non-structural proteins.

PB2-1 (SEQ ID NO: 23)
5′-GGAGACGUGGUGUUGGUAA-3′
PB2-2 (SEQ ID NO: 24)
5′-CGGGACUCUAGCAUACUUA-3′
PB1-1 (SEQ ID NO: 25)
5′-GCAGGCAAACCAUUUGAAU-3′
PB1-2 (SEQ ID NO: 26)
5′-CAGGAUACACCAUGGAUAC-3′
PB1-3 (SEQ ID NO: 27)
5′-GAUCUGUUCCACCAUUGAA-3′
PA-1 (SEQ ID NO: 28)
5′-UGCUUCAAUCCGAUGAUUG-3′
PA-1 (SEQ ID NO: 29)
5′-CGGCUACAUUGAGGGCAAG-3′
PA-2 (SEQ ID NO: 30)
5′-GCAAUUGAGGAGUGCCUGA-3′
PA-3 (SEQ ID NO: 31)
5′-UGAUCCCUGGGUUUUGCUU-3′
PA-4 (SEQ ID NO: 32)
5′-UGCUUCUUGGUUCAACUCC-3′
NP-1 (SEQ ID NO: 33)
5′-UAGAGAGAAUGGUGCUCUC-3′
NP-2 (SEQ ID NO: 34)
5′-UAAGGCGAAUCUGGCGCCA-3′
NP-3 (SEQ ID NO: 35)
5′-GGAUCUUAUUUCUUCGGAG-3′
M-1 (SEQ ID NO: 36)
5′-CCGAGGUCGAAACGUACGU-3′
M-2 (SEQ ID NO: 37)
5′-CAGAUUGCUGACUCCCAGC-3′
M-3 (SEQ ID NO: 38)
5′-UGGCUGGAUCGAGUGAGCA-3′
M-4 (SEQ ID NO: 39)
5′-GAAUAUCGAAAGGAACAGC-3′
NS-1 (SEQ ID NO: 40)
5′-CGGCUUCGCCGAGAUCAGA-3′
NS-2 (SEQ ID NO: 41)
5′-GUCCUCCGAUGAGGACUCC-3′
NS-3 (SEQ ID NO: 42)
5′-UGAUAACACAGUUCGAGUC-3′

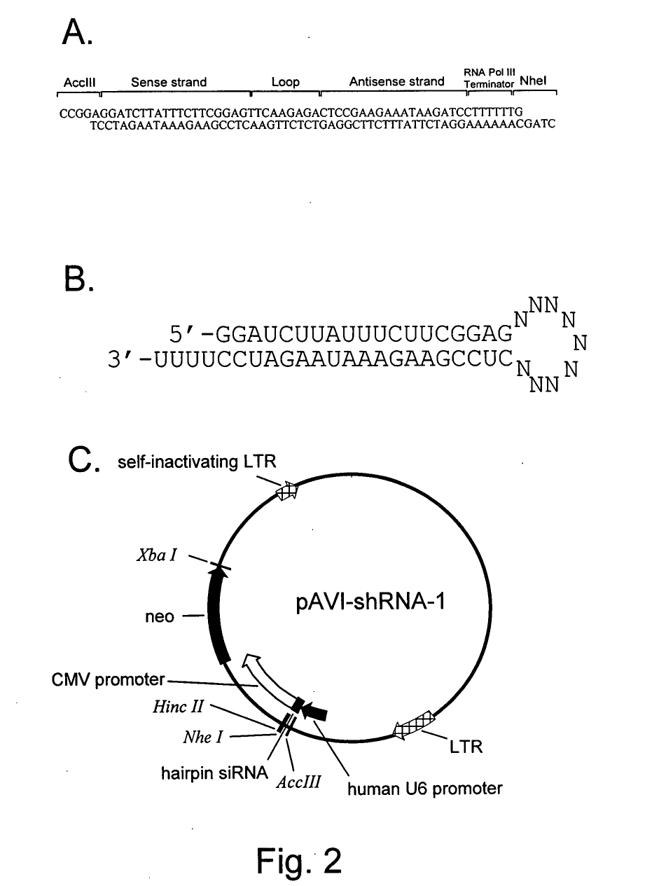

In one useful embodiment, coding sequences for shRNAs are constructed for use in producing disease resistant transgenic avians as disclosed herein wherein the shRNAs contain a sequence selected from SEQ ID NOS: 23 to 42 and the corresponding complement. See for example FIG. 2B which shows a shRNA produced from the expression cassette shown in FIG. 2A in which a shRNA employing the sequence of SEQ ID NO: 35 and its complement is shown. In this particular instance the complement and the sequence of interest (in this case SEQ ID NO: 35) are part of the same linear RNA molecule which can self-hybridize forming a hairpin (shRNA) structure. The invention, however, is not limited to shRNAs in that the sequence of interest (e.g., one of SEQ ID NOS: 23-42) and its complement can be coded for on a DNA sequence present in the genome of the transgenic avian wherein the sequence of interest and its complement are not part of the same linear RNA molecule when transcribed. For example, it is specifically contemplated that siRNAs can be constructed for SEQ ID NOS: 23 to 42 using two separate coding sequences: one sequence encoding the target sequence and one sequence encoding the complement of the target sequence, each coding sequence being under the control of different promoters. It is also contemplated that both coding sequences may be under the control of the same promoter, for example, by employing two transcription initiation sites.

Nucleotides contained in the loop of the hairpin shown in FIG. 2B are each designated as “N” since the sequence of these nucleotides is not necessarily critical to the function of the shRNA and many nucleotide sequences can be employed in such loop structures as is apparent to a practitioner of skill in the art. However, the actual sequence of the loop structure in this particular shRNA of FIG. 2B can be deduced from the coding sequence shown in FIG. 2A. FIG. 2B also shows overhanging nucleotides of the shRNA (UUUU) which are discussed herein.

In one embodiment, it is desirable to employ polynucleotide therapeutics which when in double stranded form comprise certain overhanging nucleotides. It is believed that therapeutic polynucleotides in double stranded form including one or more overhanging nucleotides may be more effective to facilitate a therapeutic effect than that of an identical double stranded polynucleotide without the overhanging nucleotides. For example, siRNA duplexes may be composed of two stands paired in a manner to have a one nucleotide or a two nucleotide or a three nucleotide 3′ overhang that overhangs one of the strands. Also, for example, shRNA duplexes may be composed of two strands paired in a manner to have a one nucleotide or a two nucleotide, or a three nucleotide, or a four nucleotide or a five nucleotide 3′ overhangs that overhang both of the strands.

The present invention provides for vectors encoding nucleotide sequences comprising therapeutic polynucleotides such as those disclosed herein. For example, nucleotide sequences of the present invention may be cloned into expression vectors which include one or more operatively-linked regulatory sequences positioned in an orientation allowing for transcription of the therapeutic polynucleotide coding sequence. In one embodiment of the invention, an RNA molecule that is antisense to the target pathogen transcript or sense to the target pathogen transcript is transcribed in vivo. In one embodiment, an RNA molecule that is antisense to the target pathogen transcript and an RNA molecule that is sense to the target pathogen transcript are transcribed in vivo in a single cell. For example, both strands of a double stranded coding region corresponding to the target pathogen transcript may be transcribed in vivo. That is, coding sequences cloned into a single vector can encode one transcript which includes both sense and antisense sequences to the target sequence (e.g., a shRNA). In one embodiment, two vectors are employed to produce the sense and anti-sense strands of a siRNA.

The present invention provides for nucleotide sequences which include one or more therapeutic polynucleotides of the invention. For example, a vector of the invention may carry sequences encoding targeting sequences directed to more than one pathogen target sequence. The targeting sequences may be directed to the same gene transcript of a certain pathogen or to different gene transcripts of a certain pathogen or to target sequences encoded in different pathogens.

In one embodiment, a vector which includes a nucleotide sequence comprising a therapeutic polynucleotide coding sequence includes promoter and/or enhancer elements in operable relationship with the therapeutic polynucleotide coding sequence. The promoter may be a constitutive or a non-constitutive promoter. Any known promoter which is useful in the present invention is contemplated for use as described herein. Useful promoters may include those such as functional portions of an avian lysozyme promoter, ovomucoid promoter, ovalbumin promoter or any promoter that is functional in an avian cell. See, for example, U.S. patent application Ser. No. 10/114,739, filed Apr. 1, 2002; U.S. patent application Ser. No. 10/856,218, filed May 28, 2004; and U.S. patent application Ser. No. 10/733,042, filed Dec. 11, 2003. The disclosures of each of these three patent applications are incorporated in their entirety herein by reference.

The promoter will include at least one functional portion of a promoter such as, but not limited to, those promoters disclosed herein. Any promoter known by a practitioner of ordinary skill in molecular biology, biochemistry, virology, bacteriology, microbiology, avian veterinary medicine and other fields related to the present invention to be sufficient to effect expression of a targeting sequence in an avian cell are within the scope of the present invention. That is, any useful RNA transcription unit or promoter may be employed in accordance with the present invention. For example, the RNA pol III transcription unit obtained from the small nuclear RNA (snRNA) U6 or from the human RNase P RNA H1 may be useful. Examples of such vector systems include, without limitation, the GeneSuppressor™ and the RNA Interference kit (commercially available from Imgenex, San Diego, Calif.). In one particularly useful embodiment, the pSIREN™ vector system (BD Biosciences, Palo Alto, Calif.) which includes the human U6 promoter is employed.

The present invention contemplates the use of self-inactivating vectors to reduce or eliminate promoter interference. For example, reducing or eliminating the interference of the promoter which is employed in transcribing RNA sequences useful in RNA interference. Production of certain self-inactivating vectors is disclosed, for example, in Flamant et al, J Gen Virol, 1993 January; 74 (Pt 1):39-46 and Ilves et al, Gene, 1996 Jun. 1; 171(2):203-8. The disclosure of each of these two references is incorporated herein in its entirety by reference.

The promoter interference (or promoter inhibition) as disclosed herein may be caused by any mechanism which results in the inhibition of transcription of the transgenic RNAi (e.g., shRNA) encoding sequences. Such mechanisms may include, but are not limited to, read-through transcription by an upstream promoter, interferon response against the transcript comprising the RNAi, promoter competition or combinations thereof.

In one embodiment, the vectors of the invention are retroviral constructs engineered to reduce or eliminate promoter interference. A promoter which inhibits transcription of a siRNA of the invention (e.g., an LTR promoter) may be inactivated, for example, by a deletion, insertion or transposition of all or part of the promoter sequence.

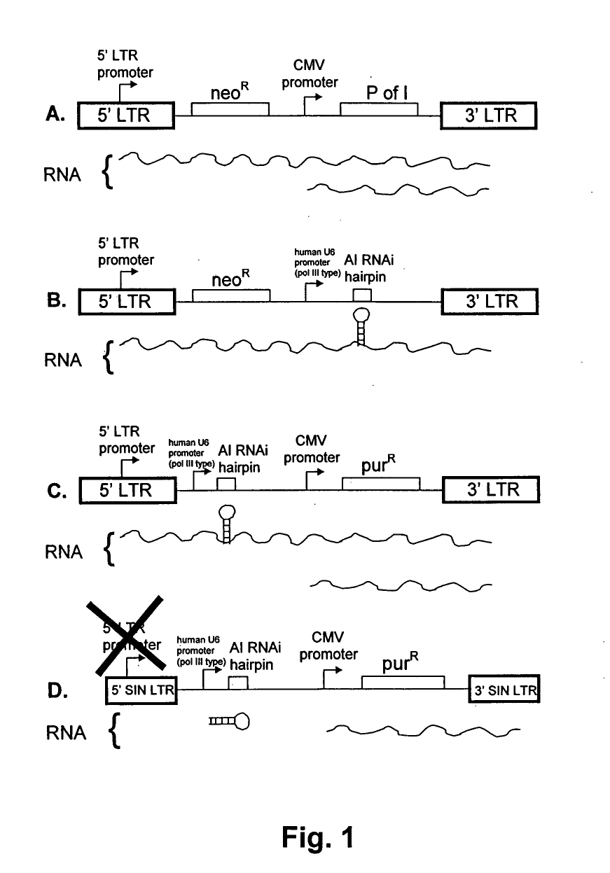

For the vector shown in FIG. 1A, the 5′ LTR promoter of the ALV produces a transcript which contains the neomycin (G418) resistance RNA fused to an RNA corresponding to the CMV promoter sequence and the sequence for the protein of interest (P of I) such as a therapeutic protein. The CMV promoter produces a transcript only for the protein of interest. These transcripts can be seen along side the bracket in FIG. 1A.

In specific embodiments, where certain promoters such as a pol III promoter (e.g., a human U6 promoter) useful for the production of RNAi transcripts in vivo are inserted into chosen vectors, inhibition of function of the pol III promoter by an upstream promoter such as a pol II promoter, for example, a 5′ LTR promoter, may occur (See, FIG. 1B). In such a case, the amount of product produced by the pol II promoter may be reduced or eliminated as shown in FIG. 1B.

In one embodiment, to construct a vector in which promoter inhibition is reduced or eliminated, an RNAi cassette (e.g., a pol III promoter driving expression of the RNAi coding sequence as shown in FIG. 1C) is inserted upstream of a selection cassette, i.e., a promoter driving expression of a selectable marker (e.g., a CMV promoter, driving expression of a puromycin resistance gene as shown in FIG. 1C). The enhancer binding region and CCAAT region of a 3′ LTR promoter of the vector are removed resulting in a 3′ SIN (self-inactivating) LTR (see FIG. 1D). Upon replication and integration of the SIN LTR viral vector, the resulting integrated 5′ SIN LTR promoter is inactivated (as is the 3′ SIN LTR promoter) due to the replication and integration process which occurs, as is understood by practitioners of ordinary skill in the art. The inactivation of the LTR provides for reduction or elimination of promoter inhibition thereby allowing for an enhanced expression of the RNAi transcript. This is merely an example of an expression vector designed to reduce or eliminate promoter interference. Other similar vector will be readily apparent to practitioner of ordinary skill in the art.

In one embodiment, all eukaryotic promoters, other than the promoter used for obtaining transcription of the therapeutic RNA, are eliminated from the retroviral construct. For example, one aspect of the invention is directed to the vector pAVI-siRNA shown in FIG. 2A in which the CMV promoter has been removed (the CMV-Neo cassette has been deleted). Without wishing to limit the invention to any particular theory or mechanism of operation, it is believed that the deletion of the CMV-neo cassette increases the titer due to shortening of the overall transgene length of the vector and may additionally reduce promoter interference of the promoter driving expression of the shRNA hairpin. Therefore, in one embodiment of the invention a retroviral vector is employed which does not contain a functional tittering cassette. In one certain aspect, the retroviral vector employed does not yield a functional promoter when integrated into the host genome aside from the promoter employed to provide transcription of the desired shRNA sequence.

The vector shown in FIG. 2 is an ALV vector (NLB) disclosed in Cosset et al (1991) J. of Virology, vol 65 p 3388-3394 which contains a cytomegalovirus (CMV) promoter driving expression of the neomycin resistance gene (neo). The CMV-neo cassette is present for the purpose of tittering preparations of retrovirus prior to transduction of cell lines or injection into chicken embryos. In one embodiment, the CMV-neo cassette is removed from pAVI-shRNA to further reduce or eliminate promoter interference, i.e., promoter interference that may be produced by the CMV promoter.

The CMV promoter is not necessarily required because, for example, production of retroviral stocks in certain instances has become routine, and as such a low titer stock is not often produced. Also, avian embryos are typically injected with the retroviral particles the same day as production (rather than freezing and storing) and if necessary the titer can be approximated by measuring the transgene content via real-time PCR in the blood DNA of hatched chicks, a process which can be termed “titer by chicken”. In addition, it has been found that when using fresh viral preps more positive chicks are consistently produced than after freezing, storing and then tittering the viral stocks.

A practitioner of ordinary skill in the field is readily able to design and construct a variety of useful expression vectors employing methods well know in the art. See, for example, Molecular Cloning: A Laboratory Manual (3^{rd} Edition) Sambrook et al. (2001) Cold Spring Harbor Laboratory Press, Cold Spring Harbor, N.Y., and Short protocols in molecular biology (5^{th} Ed.) Ausubel et al. (2002) John Wiley & Sons, New York City.

The invention provides for cells of transgenic avians which include a nucleotide sequence of the invention. For example, the invention provides for avian cells which include therapeutic polynucleotides that target RNA, for example, mRNA, expressed by an avian pathogen such as influenza virus (e.g., influenza A virus) Marek's disease virus and related viruses including, but not limited to, herpes virus of turkey.

It is understood that the description of cells of recombinant or transgenic avians disclosed herein refers not only to a particular subject cell but also to the cells of progeny or potential progeny of the avian. Because certain modifications may occur in succeeding generations due to either mutation or environmental influences, such progeny cells may not, in fact, be identical to the parent cell but are still included within the scope of the cell description as used herein. Nevertheless, cells of progeny are understood to have retained without modification the nucleotide sequence of the invention that was originally introduced into a parental avian cell. Such cells include, without limitation, cells of the skin, muscle, heart, liver, lungs, eyes, kidney, smooth muscle as well as cells from the circulatory system including reticulocytes, lymphocytes, and macrophages and cells from the reproductive system including sperm and ova.

Avian cells include, for example and without limitation, cells of a goose, pheasant, parrot, finch, hawk, crow, ratite including ostrich, emu, quail and cassowary. In one useful embodiment, the avian cells are cells of a chicken, turkey or duck.

Any useful method may be employed to incorporate nucleotide sequences of the invention into cells. Examples of such useful methods include calcium phosphate or calcium chloride co-precipitation, DEAE-dextran-mediated transfection, lipofection, use of artificial viral envelopes, ballistic particle projection, microinjection, or electroporation. In one particularly useful embodiment, nucleotide sequences of the invention are stably incorporated into the genome of avian cells. Any useful method may be employed to clone nucleotide sequences of the invention into avian genomes.

Transfection of avian cells, for example, blastodermal cells, may be accomplished by any useful method known to those of ordinary skill in the art. Vectors contemplated for introducing nucleotide sequences of the invention into an avian genome include, without limitation, retroviruses, adenoviruses, adeno-associated viruses, for example, the replication-deficient avian leucosis virus (ALV), the replication-deficient murine leukemia virus (MLV), lentivirus, herpes simplex viruses and vaccinia viruses. The invention is not limited to any particular retrovirus for the introduction of therapeutic RNAs into transgenic avians (e.g., transgenic chickens). Nor is the invention limited to the use of retroviruses for the introduction of therapeutic RNAs into transgenic avians (e.g., transgenic chickens).

Methods useful for incorporating a nucleotide sequence of the invention into the genome of an avian cell utilizing retroviruses are known in the art and are disclosed in, for example, U.S. Patent Application Publication No. 2004/0019923; U.S. Pat. No. 6,730,822; and WO 04047531, filed Jun. 10, 2004. The disclosures of this US patent application, US patent and WO publication are incorporated in their entirety herein by reference.

In one embodiment, a packaged retroviral-based vector is used to deliver the vector directly into embryonic blastodermal cells. In another embodiment, helper cells which produce retrovirus are delivered to a blastoderm. Transfection may be facilitated by mixing the virus particles with polylysine or cationic lipids which assist in passage across the cell membrane.

In one important aspect, a nucleotide sequence of the invention contained in a retrovirus (for example, and without limitation, MMLV, ALV, Lenti virus, MLV and REV), or a retroviral vector which contains components of a retrovirus such as LTRs (for example, and without limitation, MMLV, ALV, Lenti virus, MLV and REV components) useful for integration into the avian genome, is introduced into avian cells (e.g., avian germ-line cells) capable of, in whole or in part, developing into an avian such as a chicken, quail, turkey, duck, goose, pheasant, parrot, finch, hawk, crow, ratite including an ostrich, emu or cassowary.

Such cells include, without limitation, germline cells, ova, sperm cells, and embryonic cells such as blastodermal cells. Blastodermal cells may include Stage I, Stage II, Stage III, Stage IV, Stage V, Stage VI, Stage VII, Stage VIII, Stage IX, Stage X, Stage XI, and Stage XII blastoderm cells. The blastodermal cells are typically stage VII-XII cells or the equivalent thereof and preferably are near stage X. The cells useful in the present invention include, without limitation, embryonic germ (EG) cells, embryonic stem (ES) cells and primordial germ cells (PGCs). The embryonic blastodermal cells may be freshly isolated, maintained in culture, or may reside within an embryo.

In one embodiment, transformed avian cells, such as embryonic blastodermal cells, are placed in an avian egg. For example, transgenic cells of the invention may be injected into the subgerminal cavity near, for example, beneath, a recipient blastoderm in an egg.

In one embodiment, a nucleotide sequence of the invention contained in a retroviral vector may be microinjected into a germinal disc of a fertilized egg to produce a stable transgenic founder bird that passes the gene to its progeny. See, for example, U.S. patent application Ser. No. 10/679,034, filed Oct. 2, 2003, the disclosure of which is incorporated herein in its entirety by reference.

In one embodiment, vectors, for example, retroviral vectors containing a nucleotides sequence of the invention (e.g., replication-defective retroviral vector particles carrying a nucleotide sequence of the invention between the 5′ and 3′ LTRs of the retroviral rector) are injected into avian eggs, such as fertilized avian eggs. For example, avian eggs may be windowed by the method of Speksnijder, U.S. Pat. No. 5,897,998, the disclosure of which is incorporated in its entirety herein by reference, and retroviral transducing particles injected into the sub-germinal cavity of the egg. Any useful amount of transducing particles may be used. For example, an amount of transducing particles in a range of about 1×10³ to about 1×10⁹ may be used.

Once hatched, avians are raised to maturity by methods well known in the field. In one particularly useful embodiment, the transgenic avian, when matured, produces either sperm or ova comprising a nucleotide sequence or nucleotide sequences encoding one or more nucleotide sequences of the invention.

A transgenic avian has at least one cell that contains a nucleotide sequence of the invention, which includes a therapeutic polynucleotide coding sequence. However, in general a transgenic avian may have about 0.1% to about 100% or about 1% to about 100% or about 10% to about 100% or about 20% to about 100% or about 30% to about 100% or about 40% to about 100%, or about 100% of the transgenic avians cells contains a nucleotide sequence of the invention, which includes a therapeutic polynucleotide coding sequence. In one embodiment, most or all tissues and organs of a G0 transgenic avian contains a nucleotide sequence of the invention, which includes a therapeutic polynucleotide coding sequence. Typically about 100% of a G1 transgenic avians cells contains a nucleotide sequence of the invention, which includes a therapeutic polynucleotide coding sequence.

The invention contemplates any useful method of genetically transforming an avian genome with a nucleotide sequence of the invention as disclosed herein. For example, an avian blastodermal cell which includes a chromosome having a first recombination site can be transformed with a nucleotide sequence of the invention comprising a second recombination sequence. Integrase activity is introduced into the cell which specifically recognizes the first and second recombination sites under conditions such that the nucleotide sequence of the invention is inserted into the chromosome by an integrase-mediated recombination event between the first and second recombination sites. In one embodiment, an artificial chromosome is employed to produce a transgenic avian comprising a nucleotide sequence of the invention. These and other concepts which may be employed in the present invention are disclosed in, for example, U.S. patent application Ser. No. 10/790,455, filed Mar. 1, 2004 and U.S. patent application Ser. No. 10/811,136, filed Mar. 26, 2004. The disclosures of U.S. Patent Applications Nos. 10/790,455 and 10/811,136 are incorporated in their entireties herein by reference.

In one embodiment of the invention, therapeutic polynucleotides may be administered directly to an avian. The therapeutic polynucleotides may be produced by conventional methods such as methods well known to those of ordinary skill in the art including, but not limited to, production in vitro or in vivo or by chemical synthesis of the nucleotide sequences. See, for example, Tuschl et al (1999), Genes & Dev. 13: 3191-3197, the disclosure of which is incorporated herein in its entirety by reference. Useful quantities of therapeutic polynucleotides may be administered to avians by any useful method known to those of skill in the art. The therapeutic polynucleotides may be single stranded or a double stranded. Each single stranded or a double stranded therapeutic polynucleotide of the invention may be DNA, RNA, or a DNA-RNA hybrid. A therapeutic polynucleotide of the invention may include non-naturally occurring nucleotides. For example, at least one nucleotide of the therapeutic polynucleotide may be a modified nucleotide or a derivatized nucleotide. Modification or derivatization may accomplish objectives such as stabilization of the polynucleotide, enhanced cell delivery of the therapeutic polynucleotide, optimizing the hybridization of a therapeutic polynucleotide with a target sequence or enhancing the initiation of the RNAi process.

The present invention is further illustrated by the following examples, which are provided by way of illustration and should not be construed to limit the invention. The contents of all references, published patents and patents cited throughout the present application are hereby incorporated by reference in their entireties.

### EXAMPLE 1

### Production of an ALV shRNA Vector

The retroviral vector pAVI-siRNA-1 shown in FIG. 2 was constructed for use in producing an avian influenza virus resistant chicken. The vector is based on the Avian Leukosis Virus (ALV) but lacks the gag, pol and env genes, making it replication deficient. The ALV vector is also modified such that the 3′ LTR is a self-inactivating (SIN) LTR by the deletion of an enhancer region of the LTR. Briefly, as is understood by a practitioner of skill in the art, a nucleotide sequence containing the CMV-Neo cassette and the U6 promoter siRNA cassette is substituted in place of the 4.0-Lysozyme promoter and IFNa-2B coding sequence of pALV-SIN-4.0-Lys-IFNa-2B which is disclosed in U.S. patent application Ser. No. 11/699,257, filed Jan. 26, 2007, the disclosure of which is incorporated in its entirety herein by reference. Upon integration of the ALV vector in a transduced cell, the deleted enhancer of the 3′ LTR is copied to the 5′ LTR, thus greatly reducing or eliminating the promoter activity of each LTR thereby reducing or eliminating promoter interference of the internal promoter(s) used to produce the therapeutic RNA transcripts.

Retroviral particles carrying the shRNA retroviral vectors are produced by transient transfection of DF-1 cells (an immortalized chicken embryo fibroblast cell line, ATCC catalog #CRL-122203) with pAVI-siRNA-1. The shRNA retroviral vector along with a vector encoding ALV gag and pol genes and a third vector encoding the vesicular stomatitis virus (VSV) envelop gene are simultaneously transfected into the DF-1 cells. Virus is harvested from the media at 24 to 72 hours post-transfection. Typically the titer is about 10⁵. The virus can be concentrated further (up to 800 fold) by ultracentrifugation for transduction of DF-1 cells to be used for viral challenges.

Similarly, additional pAVI-siRNA vectors can be produced containing shRNA coding sequences which comprise nucleotide sequences encoding other siRNAs of SEQ ID NOs: 23 to 42. That is pAVI-siRNA vectors can be produced which contain the coding sequence for one of SEQ ID NOs: 23 to 34 or one for one of SEQ ID NOs: 36 to 42 in place of the coding sequence for SEQ ID NO: 35, as is understood by a practitioner of skill in the art.

### EXAMPLE 2

### Confirmation of Anti-Influenza A Retrovirus Activity of shRNA Encoding Vectors

DF-1 cells are susceptible to influenza infection and they can therefore serve as a test model for resistance to influenza A infection. Confirmation of influenza A resistance for DF-1 cells containing shRNA inserts are tested using two influenza viruses: A/Whooper Swan/Mongolia/244/2005 (H5N1) high pathogenicity avian influenza virus and A/chicken/Pennsylvania/1/83 (H5N2) low pathogenicity avian influenza virus. DF-1 cells are transduced with pAVI-siRNA vectors produced as disclosed in Example 1 at an approximate MOI of one.

Trypsinized DF-1 cells are diluted in M119 media containing 10% fetal bovine serum, 25 μg/ml of gentamicin, 100 units/ml of penicillin, and 2 μg/ml of amphotericin B and seeded at a density of 2×10⁶ cells/cm² in plastic tissue culture plates, consisting of a cluster of six 35 mm diameter wells. Cultures are maintained at 37° C. in a 5% CO2 atmosphere for 24 hours, and prepared for inoculation by gently washing the monolayers twice with M199 media. Six to eight serial ten-fold dilutions of virus are inoculated onto duplicate plates of DF-1 cells. Plates are incubated at 37° C. for one hour, with the inoculum being redistributed every twenty minutes. Cultures inoculated with one series of virus dilutions are overlaid with M199 media containing 0.9% Difco Bacto Agar. The replicates inoculated with the other set of dilutions are overlaid with M199 media containing 0.9% Difco Bacto Agar and 0.5 μg/ml of trypsin (T0134, Sigma Chemical Company, St. Louis, Mo.). All plates are inverted and incubated at 37° C. for 2 or 3 days, when a secondary overlay is added containing M199 media with 0.9% Difco Bacto Agar and 0.05 mg/ml of neutral red stain. Resistance to influenza virus is determined by analyzing plaque number, size, and morphology for example, through post-inoculation day 5 or 6.

### EXAMPLE 3

### Production of Transgenic Chickens and Fully Transgenic G1 Chickens Expressing shRNA

Approximately 300 White Leghorn (strain Line 0) eggs are windowed according to the procedure disclosed in U.S. Pat. No. 5,897,998, the disclosure of which is incorporated in its entirety herein by reference. Each windowed egg is injected with approximately 7×10⁴ transducing particles of pAVI-siRNA vectors produced according to Example 1 and analyzed for effectiveness according to Example 2. The eggs hatch about 21 days after injection. shRNA levels are measured by northern blot analysis of total RNA isolated from reticulocytes from chicks one week after hatch run on a 20% polyacrylamide/8 molar urea gel.

DNA is extracted from rooster sperm samples by Chelex-100 extraction (Walsh et al., 1991) to screen for G0 roosters which contained the shRNA transgene in their sperm. To identify roosters having transgene positive sperm the DNA samples are subjected to Taqman™ analysis on a Model 7700 Sequence Detector (Perkin Elmer) using primers which anneal to the viral sequence. G0 roosters with the highest levels of the transgene in their sperm samples are bred to nontransgenic SPAFAS hens by artificial insemination.

Blood DNA samples of the offspring are screened for the presence of the shRNA transgene by Taqman™ analysis as disclosed above. The sperm of the transgenic roosters identified is used for artificial insemination of nontransgenic Athens-Canadian random breed line (AC line) hens. About 50% of the offspring contain the transgene as detected by Taqman™ analysis.

G1 or G2 transgene positive birds are challenged with avian influenza A virus H5N1 and are found to be resistant to infection.

### EXAMPLE 4

### Construction of RNAi Plasmids Directed Against Marek's Disease Virus and Herpes Virus of Turkey

The oligonucleotides of Table 1 were designed based on the sequence of their corresponding target shown in Table 2. The target sequences were selected using methodologies described elsewhere herein. The oligonucleotides of Table 1 were each produced by solid phase chemical synthesis. The plasmids of Table 2 were produced by annealing the complementary oligonucleotides of Table 1, then ligating the double stranded DNA segments into linearized pSIREN vector, which includes a Human U6 promoter (BD Biosciences, Palo Alto, Calif.). Ligated plasmids were electroporated into E. coli DH5α cells. The plasmids were sequenced to verify the inserts using the U6 sequencing primer (SEQ ID NO: 15).

Each of the plasmids shown in Table 2 encode an RNA transcript which when inside of a cell, according to a non-limiting theory of the invention, will self anneal to form a small hairpin (shRNA). The plasmids shown in Table 2 designated pMDV 1 to pMDV 5 and pMDVHVT each encode a nucleotide sequence which corresponds to a segment of the MDV ICP4 gene (GenBank Accession No. M75729). pMDVHVT also encodes a nucleotide sequence which corresponds to a segment of the HVT genome (GenBank Accession No. AF282130). pFFLUCNEW encodes a luciferase shRNA effective against the luciferase target sequence designated in SEQ ID NO: 22.

TABLE 1

| | | SEQ |
|---|---|---|
| | | ID |
| NAME | SEQUENCE | NO |
| FFLUCNEW | 5′-GATCCGTGCGCTGCTGGTGCCAACTTCAAG | 1 |
| sense | AGAGTTGGCACCAGCAGCGCACTTTTTTGCTAG | |
| | CG-3′ | |
| FFLUCNEW | 5′-AATTCGCTAGCAAAAAAGTGCGCTGCTGGT | 2 |
| anti | GCCAACTCTCTTGAAGTTGGCACCAGCAGCGCA | |
| | CG-3′ | |
| MDV 1 sense | 5′-GATCCGGCGTCTCGCTGCAAACACTTCAAG | 3 |
| | AGAGTGTTTGCAGCGAGACGCCTTTTTTGCTAG | |
| | CG-3′ | |
| MDV I anti | 5′-AATTCGCTAGCAAAAAAGGCGTCTCGCTGC | 4 |
| | AAACACTCTCTTGAAGTGTTTGCAGCGAGACGC | |
| | CG-3′ | |
| MDV 2 sense | 5′-GATCCGCTCCTCAAACGGCGCAGATTTCAA | 5 |
| | GAGAATCTGCGCCGTTTGAGGAGTTTTTTGCTA | |
| | GCG-3 | |
| MDV 2 anti | 5′-AATTCGCTAGCAAAAAACTCCTCAAACGGC | 6 |
| | GCAGATTCTCTTGAAATCTGCGCCGTTTGAGGA | |
| | GCG-3 | |
| MDV 3 sense | 5′-GATCCACGGCGCAGATGAATCTGGTTCAAG | 7 |
| | AGACCAGATTCATCTGCGCCGTTTTTTTGCTAG | |
| | CG-3′ | |
| MDV 3 anti | 5′-AATTCGCTAGCAAAAAAACGGCGCAGATGA | 8 |
| | ATCTGGTCTCTTGAACCAGATTCATCTGCGCCG | |
| | TG-3′ | |
| MDV 4 sense | 5′-GATCCGTCTGGTGAGAGTTCCAGTGTTCAA | 9 |
| | GAGACACTGGAACTCTCACCAGATTTTTTGCTA | |
| | GCG-3′ | |
| MDV 4 anti | 5′-AATTCGCTAGCAAAAAATCTGGTGAGAGTT | 10 |
| | CCAGTGTCTCTTGAACACTGGAACTCTCACCAG | |
| | ACG-3′ | |
| MDV 5 sense | 5′-GATCCGGCGCTAGATCCCGATTACTTCAAG | 11 |
| | AGAGTAATCGGGATCTAGCGCCTTTTTTGCTAG | |
| | CG-3′ | |
| MDV 5 anti | 5′-AATTCGCTAGCAAAAAAGGCGCTAGATCCC | 12 |
| MDVHVT sense | 5′ GATCCGTGGAAAGCAGCACCCGATATTTCA | 13 |
| | AGAGAATATCGGGTGCTGCTTTCCATTTTTTGC | |
| | TAGCG 3′ | |
| MDVHVT anti | 5′ AATTCGCTAGCAAAAAATGGAAAGCAGCAC | 14 |
| | CCGATATTCTCTTGAAATATCGGGTGCTGCTTT | |
| | CCACG 3′ | |
| U6 Primer | 5′-GAGGGCCTATTTCCCATGAT-3′ | 15 |

TABLE 2

| | | Plasmid Encoding |
|---|---|---|
| | | Corresponding |
| Target Name | Target Sequence | shRNA |
| Target- | gtgcgctgctggtgccaac | pFFLUCNEW |
| Luciferase | SEQ ID NO: 16 | |
| Target- | ggcgtctcgctgcaaacac | pMDV 1 |
| MDV No.1 | SEQ ID NO: 17 | |
| Target- | ctcctcaaacggcgcagat | pMDV 2 |
| MDV No.2 | SEQ ID NO: 18 | |
| Target- | acggcgcagatgaatctgg | pMDV 3 |
| MDV No.3 | SEQ ID NO: 19 | |
| Target- | tctggtgagagttccagtg | pMDV 4 |
| MDV No.4 | SEQ ID NO: 20 | |
| Target- | ggcgctagatcccgattac | pMDV 5 |
| MDV No.5 | SEQ ID NO: 21 | |
| Target- | tggaaagcagcacccgatat | pMDVHVT |
| MDVHVT | SEQ ID NO: 22 | |

### EXAMPLE 5

### Effectiveness of RNAi Plasmids Directed Against Luciferase Expression

Two cell lines, human breast carcinoma cells (MCF 7) and chicken fibroblast cells (DF-1) were cotransfected. Each cotransfection was performed with three plasmids: 1. pFFLUCNEW which includes the human U6 promoter that drives expression of a luciferase shRNA; 2. pGWIZ which includes a CMV promoter that drives expression of firefly luciferase; and 3. pCMV-hIFN, which includes a CMV promoter that drives expression of human interferon α-2b. The purpose of the pCMV-hIFN plasmid is to normalize for transfection efficiency. For negative control transfections, pSIREN (BD Biosciences) was used instead of pFFLUCNEW.

For each transfection, 10⁵ cells per well of a 24-well plate were transfected with 1) 250 ng of pFFLUNEW; 2) 50, 150, or 250 ng of pGWIZ; and 3) 500 ng pCMV-hIFN. Quantitative analysis of interferon levels and luciferase activity were determined for each of the recombinant cell lines using standard methodologies well known in the field of biochemistry.

Graph 1 shows two sets of bars representing two transfection experiments performed on two successive days. Each transfection experiment was performed in duplicate, the mean values of which are represented by individual bars. The data demonstrates an active RNAi effect produced by pFFLUCNEW against luciferase in MCF 7 cells and DF1 cells.

### EXAMPLE 6

### Inhibition of MDV and HVT Infection by RNAi

Chicken embryo fibroblasts were transfected with each of the MDV RNAi plasmids listed in Table 2, except for pMDV 1 which was not tested. The cells were simultaneously infected with either herpes virus of turkey (HVT) (Table 3) or RB1B which is a particularly virulent strain of MDV (Table 4). For a negative control, only MDV or HVT was transfected into the cell line. The luciferase RNAi plasmid, pFFLUCNEW, was transfected as a background control for transfection efficiency both in HVT and MDV experiments.

For each transfection, 10 ug of total genomic DNA isolated from HVT-infected chicken embryo fibroblast (CEF) or RB1B MDV-infected chicken embryo fibroblast (CEF) and 11.0 ug of each RNAi plasmid type to be transfected were added to a 5 ml polystyrene tube in 438 ul of sterile water. 62 ul of 2M calcium chloride (CaCl₂) was added and using a sterile 1 ml pipet, 500 ul of 2×HBSP (hepes-buffered saline phosphate) was slowly added to the reaction allowing 10-15 bubbles to blow from the tip of the pipet to mix the solution. A fine white precipitate formed within minutes after the HBSP addition. Within 15 minutes of precipitate formation, each transfection reaction was divided into two 60 mm dishes (500 ul per dish) holding 5 ml of CEF cells at 7×10⁵ cells/ml.

The dishes were incubated at 37° C. and 5% CO₂ for 3.5 to 4.5 hours after which time the cells were exposed to a glycerol shock procedure as follows:

a. the media was removed and the cell monolayers were washed with 4 ml of incomplete media (M199+antibiotics);

b. the incomplete media was removed and 2 ml of glycerol shock solution (15% glycerol in 1×PBS) was added to each monolayer;

c. after 2 min, the glycerol was removed and each monolayer was washed with 4 ml complete media (M199+antibiotics+3% calf serum);

d. the wash media was removed and 5 ml of fresh complete media was added;

e. the dishes were incubated at 37° C. and 5% CO₂ for 1 week or until plaques were visible.

Cells are not firmly attached and monolayers are not confluent during the glycerol shock process. As a result there was a certain amount of cell loss during this procedure. In addition, the glycerol shock was performed in a gentle manner because the cells are fragile and susceptible to mechanical damage.

TABLE 3

| | | | Overall | Normalized |
|---|---|---|---|---|
| | | | Percent^{a} | Percent^{b} |
| | | | Reduction | Reduction in |
| | #plaques | Average | in Number | Number of |
| Cotransfection | dish A, B | #plaques | of Plaques | Plaques |
| HVT only | 470, 455 | 462 | | |
| HVT + pFFLUCNEW | 193, 221 | 207 | 55 | |
| HVT + pMDV 2 | 205, 194 | 200 | 57 | 3 |
| HVT + pMDV 3 | 194, 194 | 194 | 58 | 6 |
| HVT + pMDV 4 | 195, 188 | 192 | 58 | 7 |
| HVT + pMDV 5 | 199, 183 | 191 | 59 | 8 |
| HVT + pMDVHVT | 46, 50 | 48 | 90 | 77 |

^{a}= 100 − (avg. # plaques experimental/average # plaques for HVT) × 100
^{b}= 100 − (avg. # plaques experimental/average # plaques for HVT + luciferase) × 100

The results obtained when the CEFs were transfected with HVT are shown in Table 3. The results show a significant reduction in the number of plaques for pMDVHVT indicating a substantial reduction in cellular HVT infection.

TABLE 4

| | | Average | Overall | Normalized |
|---|---|---|---|---|
| | Number of | Number | Percent^{c} | Percent^{d} |
| | Plaques - | Of | Reduction | Reduction in |
| | Dish - A, | Plaques | in Number | Number of |
| Cotransfection | B | Per Dish | of Plaques | Plaques |
| MDV only | 140, 214 | 177 | | |
| MDV + pFFLUCNEW | 60, 60 | 60 | 66 | |
| MDV + pMDV 2 | 6, 14 | 10 | 94 | 83 |
| MDV + pMDV 3 | 19, 16 | 18 | 90 | 70 |
| MDV + pMDV 4 | 5, 4 | 4 | 98 | 93 |
| MDV + pMDV 5 | 3, 7 | 5 | 97 | 92 |
| MDV + pMDV/HVT | 9, 9 | 9 | 95 | 85 |

^{c}100 − (avg. # plaques experimental/average # plaques for MDV) × 100
^{d}100 − (avg. # plaques experimental/average # plaques for MDV + luciferase) × 100

The results obtained when the CEFs were transfected with the RB1B pathotype of MDV are shown in Table 4. Each of the pMDV tested reduced MDV cellular infection significantly.

These results demonstrate that RNAi interference directed against HTV and MDV is highly effective.

### EXAMPLE 7

### Construction of Vector Suitable for Transgenesis and Production of Transduction Particles

The lacZ gene of pNLB, a replication-deficient avian leukosis virus (ALV)-based vector (Cosset et al., 1991, J. Virol. 65:3388-3394, the disclosure which is incorporated in its entirety herein by reference) is replaced with an expression cassette which includes a human U6 promoter operably linked to a therapeutic polynucleotide coding sequence represented by SEQ ID NO: 3 annealed to its complement represented by SEQ ID NO: 4 to produce pNLB-U6-MDV.

Transduction particles are produced as described in Cosset supra with the following exceptions. Two days after transfection of the retroviral vector pNLB-U6-MDV into 9×10⁵ Sentas, virus is harvested in fresh media for 6 to 16 hours and filtered. All of the media is used to transduce 3×10⁶ Isoldes in three 100 mm plates with polybrene added to a final concentration of 4 μg/ml. The following day the media is replaced with media containing 50 μg/ml phleomycin, 50 μg/ml hygromycin and 200 μg/ml G418 (Sigma).

After 10-12 days, single G418^{r} colonies are isolated and transferred to 24-well plates. After 7-10 days, titers from each colony are determined by transduction of Sentas followed by G418 selection. Colonies giving high titers are chosen for virus propagation. Certain aspects of this protocol are disclosed in Allioli et al (1994) Dev. Biol. 165:30-37, the disclosure of which is incorporated herein by reference.

### EXAMPLE 8

### Production of Transgenic Chickens and Fully Transgenic G1 Chickens Expressing shRNA

Approximately 300 White Leghorn (strain Line 0) eggs are windowed according to the procedure disclosed in U.S. Pat. No. 5,897,998, the disclosure of which is incorporated in its entirety herein by reference. Each windowed egg is injected with approximately 7×10⁴ transducing particles of pNLB-U6-MDV produced according to Example 7. The eggs hatch about 21 days after injection. shRNA levels are measured by northern blot analysis of total RNA isolated from reticulocytes from chicks one week after hatch run on a 20% polyacrylamide/8 molar urea gel.

DNA is extracted from rooster sperm samples by Chelex-100 extraction (Walsh et al., 1991) to screen for G0 roosters which contained the shRNA transgene in their sperm. The DNA samples are subjected to Taqman™ analysis on a Model 7700 Sequence Detector (Perkin Elmer) using primers which anneal to the viral sequence and probes which hybridize to the neomycin resistance coding sequence to detect the transgene. G0 roosters with the highest levels of the transgene in their sperm samples are bred to nontransgenic SPAFAS hens by artificial insemination.

Blood DNA samples of the offspring are screened for the presence of the shRNA transgene by Taqman™ analysis as disclosed above. The sperm of the transgenic roosters identified is used for artificial insemination of nontransgenic Athens-Canadian random breed line (AC line) hens. About 50% of the offspring contain the transgene as detected by Taqman™ analysis.

G2 birds are challenged with MDV. Approximately 90% of the birds show resistance to MDV.

### EXAMPLE 9

### Production of Transgenic Ducks and Fully Transgenic G1 Ducks Expressing shRNA

Approximately 400 Cayuga duck (strain Line 0) eggs are windowed essentially as described in Speksnijder, U.S. Pat. No. 5,897,998. Each windowed egg is injected with approximately 7×10⁴ transducing particles of retroviral pNLB-U6-MDV produced according to Example 7. The eggs hatch about 21 days after injection. shRNA levels are measured by northern blot analysis of total RNA isolated from reticulocytes from chicks one week after hatch run on a 20% polyacrylamide/8 molar urea gel.

DNA is extracted from male duck sperm samples by Chelex-100 extraction (Walsh et al., 1991) to screen for G0 male ducks which contained the shRNA transgene in their sperm. The DNA samples are subjected to Taqman™ analysis on a Model 7700 Sequence Detector (Perkin Elmer) using primers which anneal to the viral sequence and probes which hybridize to the neomycin resistance coding sequence to detect the transgene. G0 ducks with the highest levels of the transgene in their sperm samples are bred to nontransgenic Cayuga ducks by artificial insemination.

Blood DNA samples of the offspring are screened for the presence of the shRNA transgene by Taqman™ analysis. The sperm of the transgenic male ducks identified is used for artificial insemination of nontransgenic Muscovey ducks. About 50% of the offspring contain the transgene as detected by Taqman™ analysis.

G2 birds are challenged with MDV. Approximately 85% of the birds show resistance to MDV.

### EXAMPLE 10

### Production of Transgenic Turkeys and Fully Transgenic G1 Turkeys Expressing shRNA

Approximately 300 white turkey (strain Line 0) eggs are windowed as disclosed in U.S. Pat. No. 5,897,998. Each windowed egg is injected with approximately 7×10⁴ transducing particles of a pNLB-U6-MDVHVT vector which is produced essentially as describe in Example 7 except that the U6 promoter is operably linked to a therapeutic polynucleotide coding sequence represented by SEQ ID NO: 13 annealed to the nucleotide sequence represented by SEQ ID NO: 14. The eggs hatch about 21 days after injection. shRNA levels are measured by northern blot analysis of total RNA isolated from reticulocytes from chicks one week after hatch run on a 20% polyacrylamide/8 molar urea gel.

DNA is extracted from turkey sperm samples by Chelex-100 extraction (Walsh et al., 1991) to screen for G0 turkeys which contained the shRNA transgene in their sperm. The DNA samples are subjected to Taqman™ analysis on a Model 7700 Sequence Detector (Perkin Elmer) using primers which anneal to the viral sequence and probes which hybridize to the neomycin resistance coding sequence to detect the transgene. G0 turkeys with the highest levels of the transgene in their sperm samples are bred to nontransgenic white turkeys by artificial insemination.

Blood DNA samples of the offspring are screened for the presence of the shRNA transgene by Taqman™ analysis. The sperm of the transgenic male turkeys identified is used for artificial insemination of nontransgenic black turkeys. About 50% of the offspring contain the transgene as detected by Taqman™ analysis.

G2 birds are challenged with MDV. Approximately 90% of the birds show resistance to HVT.

While this invention has been described with respect to various specific examples and embodiments, it is to be understood that the invention is not limited thereto and that it can be variously practiced with the scope of the following claims.

What is claimed is:

1. A transgenic avian containing in its genome a recombinant nucleotide sequence encoding a therapeutic polynucleotide comprising a nucleotide sequence complementary to a conserved nucleotide sequence in genetic material of influenza virus.

2. The transgenic avian of claim 1 wherein the therapeutic polynucleotide facilitates RNA interference in a cell of the transgenic avian.

3. The transgenic avian of claim 1 wherein the therapeutic polynucleotide is RNA.

4. The transgenic avian of claim 1 wherein the therapeutic polynucleotide comprises a first nucleotide sequence and a second nucleotide sequence wherein the second nucleotide sequence has substantially the same length as the first nucleotide sequence and is complementary to the first nucleotide sequence.

5. The transgenic avian of claim 4 wherein the first nucleotide sequence hybridizes to the second nucleotide sequence.

6. The transgenic avian of claim 4 wherein the second nucleotide sequence is longer than the first nucleotide sequence by one of: one nucleotide, two nucleotides, three nucleotides and four nucleotides.

7. The transgenic avian of claim 1 wherein the therapeutic polynucleotide comprises a first nucleotide sequence attached to a second nucleotide sequence by a loop sequence wherein the second nucleotide sequence has substantially the same length as the first nucleotide sequence and is complementary to the first nucleotide sequence.

8. The transgenic avian of claim 7 wherein the first nucleotide sequence hybridizes to the second nucleotide sequence to form a hairpin.

9. The transgenic avian of claim 7 wherein the second nucleotide sequence is longer than the first nucleotide sequence by one of: one nucleotide, two nucleotides, three nucleotides and four nucleotides.

10. The transgenic avian of claim 1 wherein the therapeutic polynucleotide is single stranded or is included in a double stranded molecule.

11. The transgenic avian of claim 1 wherein the therapeutic polynucleotide is between about 10 nucleotides and about 200 nucleotides in length.

12. The transgenic avian of claim 1 wherein the therapeutic polynucleotide is between about 15 nucleotides and about 35 nucleotides in length.

13. The transgenic avian of claim 1 wherein the conserved nucleotide sequence is about 10 to about 50 nucleotides in length.

14. The transgenic avian of claim 1 wherein the conserved nucleotide sequence is about 15 to about 25 nucleotides in length.

15. The transgenic avian of claim 1 wherein the therapeutic polynucleotide is at least about 90% complementary to the conserved nucleotide sequence in genetic material of influenza virus A.

16. The transgenic avian of claim 1 wherein the therapeutic polynucleotide is at least about 95% complementary to the conserved nucleotide sequence in genetic material of influenza virus A.

17. The transgenic avian of claim 1 wherein the therapeutic polynucleotide is present in a cell of the avian and is included in a complex which facilitates cleavage of a nucleotide sequence in genetic material of an avian influenza virus.

18. The transgenic avian of claim 17 wherein the complex is a RISC complex.

19. The transgenic avian of claim 1 wherein the therapeutic polynucleotide inhibits replication of the avian influenza virus.

20. The transgenic avian of claim 1 wherein the recombinant nucleotide sequence comprises at least one of a promoter and an enhancer in operable relationship to the therapeutic polynucleotide sequence.

21. The transgenic avian of claim 20 wherein the promoter is effective to express the therapeutic polynucleotide in an avian cell.

22. The transgenic avian of claim 20 wherein the promoter comprises a polymerase III promoter or a functional portion thereof.

23. The transgenic avian of claim 1 wherein the recombinant nucleotide sequence is integrated in a chromosome.

24. The transgenic avian of claim 1 wherein the nucleotide sequence encoding a therapeutic polynucleotide comprises a nucleotide sequence substantially identical to a conserved nucleotide sequence present in a coding sequence of a gene selected from the group consisting of NP, PA, PB1, PB2, M and NS.

25. The transgenic avian of claim 1 wherein the nucleotide sequence encoding a therapeutic polynucleotide comprises a nucleotide sequence selected from the group consisting of SEQ ID NO: 23, SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; of SEQ ID NO: 34; SEQ ID NO: 35; SEQ ID NO: 36; SEQ ID NO: 37; SEQ ID NO: 38; SEQ ID NO: 39; SEQ ID NO: 40; SEQ ID NO: 41; and SEQ ID NO: 42.

26. The avian of claim 1 wherein the avian is selected from the group consisting of a chicken, a turkey, a duck and a quail.

27. The avian of claim 1 wherein the avian is a chicken.

28. A transgenic avian containing a nucleotide sequence in its genome encoding a therapeutic polynucleotide comprising a nucleotide sequence complementary to a nucleotide sequence in genetic material of an avian influenza virus wherein the therapeutic polynucleotide comprises a first nucleotide sequence attached to second nucleotide sequence by a loop sequence wherein the second nucleotide sequence is complementary to at least a portion of the first nucleotide sequence.

29. A method for producing a transgenic avian comprising:
providing a recombinant nucleotide sequence encoding a therapeutic polynucleotide comprising a nucleotide sequence substantially complementary to a conserved nucleotide sequence in genetic material of an avian influenza virus wherein the therapeutic polynucleotide facilitates RNA interference in an avian cell;
introducing the recombinant nucleotide sequence into avian cells capable of developing into a mature avian; and
obtaining a mature transgenic avian,
thereby producing a transgenic avian.

30. The method of claim 29 wherein the transgenic avian produces either sperm or ova comprising the recombinant nucleotide sequence.

31. The method of claim 29 wherein the transgenic avian is protected against infection by influenza.

32. The method of claim 29 wherein the recombinant nucleotide sequence encoding a therapeutic polynucleotide comprises a conserved nucleotide sequence substantially identical to a nucleotide sequence present in a coding sequence of a gene selected from the group consisting of NP, PA, PB1, PB2, M and NS.

33. The method of claim 29 wherein the recombinant nucleotide sequence encoding a therapeutic polynucleotide comprises a nucleotide sequence selected from the group consisting of SEQ ID NO: 23, SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; of SEQ ID NO: 34; SEQ ID NO: 35; SEQ ID NO: 36; SEQ ID NO: 37; SEQ ID NO: 38; SEQ ID NO: 39; SEQ ID NO: 40; SEQ ID NO: 41; and SEQ ID NO: 42.


SEQUENCE LISTING (33 sequences)

SEQ ID NO: 1 - DNA, 65 bp, Artificial Sequence
  Nucleotide Sequence From Marek′s Disease Virus
gatccgtgcgctgctggtgccaacttcaagagagttggcaccagcagcgcacttttttgctagcg

SEQ ID NO: 2 - DNA, 65 bp, Artificial Sequence
  Nucleotide Sequence From Marek′s Disease Virus
aattcgctagcaaaaaagtgcgctgctggtgccaactctcttgaagttggcaccagcagcgcacg

SEQ ID NO: 3 - DNA, 65 bp, Artificial Sequence
  Nucleotide Sequence From Marek′s Disease Virus
gatccggcgtctcgctgcaaacacttcaagagagtgtttgcagcgagacgccttttttgctagcg

SEQ ID NO: 4 - DNA, 65 bp, Artificial Sequence
  Nucleotide Sequence From Marek′s Disease Virus
aattcgctagcaaaaaaggcgtctcgctgcaaacactctcttgaagtgtttgcagcgagacgccg

SEQ ID NO: 5 - DNA, 66 bp, Artificial Sequence
  Nucleotide Sequence From Marek′s Disease Virus
gatccgctcctcaaacggcgcagatttcaagagaatctgcgccgtttgaggagttttttgctagcg

SEQ ID NO: 6 - DNA, 66 bp, Artificial Sequence
  Nucleotide Sequence From Marek′s Disease Virus
aattcgctagcaaaaaactcctcaaacggcgcagattctcttgaaatctgcgccgtttgaggagcg

SEQ ID NO: 7 - DNA, 65 bp, Artificial Sequence
  Nucleotide Sequence From Marek′s Disease Virus
gatccacggcgcagatgaatctggttcaagagaccagattcatctgcgccgtttttttgctagcg

SEQ ID NO: 8 - DNA, 65 bp, Artificial Sequence
  Nucleotide Sequence From Marek′s Disease Virus
aattcgctagcaaaaaaacggcgcagatgaatctggtctcttgaaccagattcatctgcgccgtg

SEQ ID NO: 9 - DNA, 66 bp, Artificial Sequence
  Nucleotide Sequence From Marek′s Disease Virus
gatccgtctggtgagagttccagtgttcaagagacactggaactctcaccagattttttgctagcg

SEQ ID NO: 10 - DNA, 66 bp, Artificial Sequence
  Nucleotide Sequence From Marek′s Disease Virus
aattcgctagcaaaaaatctggtgagagttccagtgtctcttgaacactggaactctcaccagacg

SEQ ID NO: 11 - DNA, 65 bp, Artificial Sequence
  Nucleotide Sequence From Marek′s Disease Virus
gatccggcgctagatcccgattacttcaagagagtaatcgggatctagcgccttttttgctagcg

SEQ ID NO: 12 - DNA, 65 bp, Artificial Sequence
  Nucleotide Sequence From Marek′s Disease Virus
aattcgctagcaaaaaaggcgctagatcccgattactctcttgaagtaatcgggatctagcgccg

SEQ ID NO: 13 - DNA, 68 bp, Artificial Sequence
  Nucleotide Sequence From Marek′s Disease Virus
gatccgtggaaagcagcacccgatatttcaagagaatatcgggtgctgctttccattttttgctagcg

SEQ ID NO: 14 - DNA, 68 bp, Artificial Sequence
  Nucleotide Sequence From Marek′s Disease Virus
aattcgctagcaaaaaatggaaagcagcacccgatattctcttgaaatatcgggtgctgctttccacg

SEQ ID NO: 15 - DNA, 20 bp, Artificial Sequence
  Nucleotide Sequence From Marek′s Disease Virus
gagggcctatttcccatgat

SEQ ID NO: 16 - DNA, 19 bp, Artificial Sequence
  Nucleotide Sequence From Marek′s Disease Virus
gtgcgctgctggtgccaac

SEQ ID NO: 17 - DNA, 19 bp, Artificial Sequence
  Nucleotide Sequence From Marek′s Disease Virus
ggcgtctcgctgcaaacac

SEQ ID NO: 18 - DNA, 19 bp, Artificial Sequence
  Nucleotide Sequence From Marek′s Disease Virus
ctcctcaaacggcgcagat

SEQ ID NO: 19 - DNA, 19 bp, Artificial Sequence
  Nucleotide Sequence From Marek′s Disease Virus
acggcgcagatgaatctgg

SEQ ID NO: 20 - DNA, 19 bp, Artificial Sequence
  Nucleotide Sequence From Marek′s Disease Virus
tctggtgagagttccagtg

SEQ ID NO: 21 - DNA, 19 bp, Artificial Sequence
  Nucleotide Sequence From Marek′s Disease Virus
ggcgctagatcccgattac

SEQ ID NO: 22 - DNA, 20 bp, Artificial Sequence
  Nucleotide Sequence From Marek′s Disease Virus
tggaaagcagcacccgatat

SEQ ID NO: 23 - DNA, 19 bp, Artificial Sequence
  Nucleotide Sequence From Influenza Virus
ttcaatggtggaacagatc

SEQ ID NO: 24 - DNA, 19 bp, Artificial Sequence
  Nucleotide Sequence From Influenza Virus
attcaaatggtttgcctgc

SEQ ID NO: 25 - DNA, 19 bp, Artificial Sequence
  Nucleotide Sequence From Influenza Virus
gtatccatggtgtatcctg

SEQ ID NO: 26 - DNA, 19 bp, Artificial Sequence
  Nucleotide Sequence From Influenza Virus
tcaggcactcctcaattgc

SEQ ID NO: 27 - DNA, 19 bp, Artificial Sequence
  Nucleotide Sequence From Influenza Virus
aagcaaaacccagggatca

SEQ ID NO: 28 - DNA, 19 bp, Artificial Sequence
  Nucleotide Sequence From Influenza Virus
taagtatgctagagtcccg

SEQ ID NO: 29 - DNA, 19 bp, Artificial Sequence
  Nucleotide Sequence From Influenza Virus
ttaccaacaccacgtctcc

SEQ ID NO: 30 - DNA, 19 bp, Artificial Sequence
  Nucleotide Sequence From Influenza Virus
tggcgccagattcgcctta

SEQ ID NO: 31 - DNA, 19 bp, Artificial Sequence
  Nucleotide Sequence From Influenza Virus
gagagcaccattctctcta

SEQ ID NO: 32 - DNA, 19 bp, Artificial Sequence
  Nucleotide Sequence From Influenza Virus
acgtacgtttcgacctcgg

SEQ ID NO: 33 - DNA, 19 bp, Artificial Sequence
  Nucleotide Sequence From Influenza Virus
tgctcactcgatccagcca